SEQUENCE LISTING (52 sequences)

SEQ ID NO: 1 - DNA, 20 bp, artificial sequence
  crRNA DNA equivalent
gttcaagaagcacacgactg

SEQ ID NO: 2 - DNA, 20 bp, artificial sequence
  crRNA DNA equivalent
gttaactgctcactggatgg

SEQ ID NO: 3 - DNA, 20 bp, artificial sequence
  crRNA DNA equivalent
ggagccggctgagagaagtt

SEQ ID NO: 4 - DNA, 20 bp, artificial sequence
  crRNA DNA equivalent
gtggagataatctgtcctaa

SEQ ID NO: 5 - DNA, 20 bp, artificial sequence
  crRNA DNA equivalent
agtccctatcccatcatcca

SEQ ID NO: 6 - DNA, 20 bp, artificial sequence
  crRNA DNA equivalent
tgagcattgcagactaatct

SEQ ID NO: 7 - DNA, 20 bp, artificial sequence
  crRNA DNA equivalent
aagtccctatcccatcatcc

SEQ ID NO: 8 - DNA, 20 bp, artificial sequence
  crRNA DNA equivalent
tcttggatatttgtccctga

SEQ ID NO: 9 - DNA, 20 bp, artificial sequence
  crRNA DNA equivalent
gttgggaaataaactgtcta

SEQ ID NO: 10 - DNA, 20 bp, artificial sequence
  crRNA DNA equivalent
ggtggataacgccctccaat

SEQ ID NO: 11 - DNA, 20 bp, artificial sequence
  crRNA DNA equivalent
gtggataacgccctccaatc

SEQ ID NO: 12 - DNA, 20 bp, artificial sequence
  crRNA DNA equivalent
ctgggagttacccgattgga

SEQ ID NO: 13 - DNA, 20 bp, artificial sequence
  crRNA DNA equivalent
cctccaatcgggtaactccc

SEQ ID NO: 14 - DNA, 20 bp, artificial sequence
  crRNA DNA equivalent
atccaccttccactgtactt

SEQ ID NO: 15 - DNA, 20 bp, artificial sequence
  crRNA DNA equivalent
ttcaactgctcatcagatgg

SEQ ID NO: 16 - DNA, 20 bp, artificial sequence
  crRNA DNA equivalent
gatttcaactgctcatcaga

SEQ ID NO: 17 - DNA, 20 bp, artificial sequence
  crRNA DNA equivalent
tgggatagaagttattcagc

SEQ ID NO: 18 - DNA, 20 bp, artificial sequence
  crRNA DNA equivalent
attcagcaggcacacaacag

SEQ ID NO: 19 - DNA, 20 bp, artificial sequence
  crRNA DNA equivalent
ggccaaagtacagtggaagg

SEQ ID NO: 20 - DNA, 20 bp, artificial sequence
  crRNA DNA equivalent
gtcctcggggcatgttccga

SEQ ID NO: 21 - DNA, 20 bp, artificial sequence
  rRNA DNA equivalent
tcctcggggcatgttccgag

SEQ ID NO: 22 - DNA, 20 bp, artificial sequence
  crRNA DNA equivalent
aggcatcggaaaatccacag

SEQ ID NO: 23 - DNA, 20 bp, artificial sequence
  crRNA DNA equivalent
ctcaggttgggtgcgtctga

SEQ ID NO: 24 - DNA, 20 bp, artificial sequence
  crRNA DNA equivalent
acgagatgcctgaacaaacc

SEQ ID NO: 25 - DNA, 20 bp, artificial sequence
  crRNA DNA equivalent
acctgagtcccattttccaa

SEQ ID NO: 26 - DNA, 20 bp, artificial sequence
  crRNA DNA equivalent
tcagccatcactaagacccc

SEQ ID NO: 27 - DNA, 20 bp, artificial sequence
  crRNA DNA equivalent
caaaccaggggtcttagtga

SEQ ID NO: 28 - DNA, 20 bp, artificial sequence
  crRNA DNA equivalent
ctaagacccctggtttgttc

SEQ ID NO: 29 - DNA, 20 bp, artificial sequence
  crRNA DNA equivalent
tcaggcatctcgtccaaatg

SEQ ID NO: 30 - DNA, 2868 bp, Mus musculus
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 31 - DNA, 2347 bp, Mus musculus
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagggaagcggagagggcagaggaagtctgctaacatgcggtgacgtcgaggagaatcctggacctatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgagatga

SEQ ID NO: 32 - DNA, 2910 bp, Mus musculus
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattcttcctatgtgcgcccgctgtcagtggccctgggggagacggccaggatttcctgtggacgacaggcccttggaagtagagctgttcagtggtatcaacataggccaggccaggcccctatattgctcatttataataatcaagaccggccctcagggatccctgagcgattctctggcacccctgatattaattttgggaccagggccaccctgaccatcagcggggtcgaagccggggatgaagccgactattactgtcacatgtgggatagtagaagtggcttcagttggtctttcggcggggcgaccaggctgaccgtcctacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtgcagctgcaggagtcgggcccaggactggtgaaaccttcggagaccctgtccgtcacctgcagtgtctctggagattccatgaataattactactggacttggatccggcagtcccccggaaagggactggagtggataggctatatctctgacagagaatcagcgacttacaacccctccctcaatagtcgagtcgtcatatcacgagacacgtcgaaaaaccaattgtccctaaaattaaactccgtcacccctgcggacacggccgtctattactgtgcgacagcgcgccgaggacagaggatttatggagtggtttcctttggagagttcttctactactactccatggacgtctggggcaaggggaccacggtcaccgtctcctcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 33 - DNA, 2856 bp, Mus musculus
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattctgacatccagatgacccagtctccatcctccctgtctgcctctgtgggagataccgtcactatcacttgccaggcaaacggctacttaaattggtatcaacagaggcgagggaaagccccaaaactcctgatctacgatgggtccaaattggaaagaggggtcccatcaaggttcagtggaagaagatgggggcaagaatataatctgaccatcaacaatctgcagcccgaagacattgcaacatatttttgtcaagtgtatgagtttgtcgtccctgggaccagactggatttgaaacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccaattgttacagtctggggcagcggtgacgaagcccggggcctcagtgagagtctcctgcgaggcttctggatacaacattcgtgactactttattcattggtggcgacaggccccaggacagggccttcagtgggtgggatggatcaatcctaagacaggtcagccaaacaatcctcgtcaatttcagggtagagtcagtctgactcgacacgcgtcgtgggactttgacacattttccttttacatggacctgaaggcactaagatcggacgacacggccgtttatttctgtgcgcgacagcgcagcgactattgggatttcgacgtctggggcagtggaacccaggtcactgtctcgtcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 34 - DNA, 2901 bp, Mus musculus
gcatagctagcgctcttcagtaagaatggcctctccaggtctttatttttaacctttgttatggagttttctgagcattgcagactaatcttggatatttgtccctgagggagccggctgagagaagttaagagtagcaacaaggaaatagcagggtgtagagggatctcctgtctgacaggaggcaagaagacagattcttacccctccatttctcttttatccctctctggtcctcagagagttagaacttgtttattgcagcttataatggttacaaataaagcaatagcatcacaaatttcacaaataaagcatttttttcactgcattctagttgtggtttgtccaaactcatcaatgtatcttatcatgtctggtcgacagtatgcagagggctgtatccactggagaggatgaagtcactgagttggaaaacagaacaggacaggcacctaacaagtggttgctatagcccactgttacccttttacatgtataggctcaggataagcagtgatactgtgaggtttatgtgtgagaacatcacagtataaacacatctcaatagaggtcttagagatcagcacaattagtgagaagtcataaacagtagatactataaggcataggctcagctacctagggtcaggtatctgtgtaaatctgattgtgtatcaggtttagatcaatatgacttagggaggcgagtcatatgcaaatctaagaagactttagagaagaaatctgaggctcacctcacataacagcaagagagtgtccggttagtctcaaggaagactgagacacagtcttagatatcaccatgggatggtcatgtatcatcctttttctagtagcaactgcaaccggtgtacattcttccgatatatctgtggccccaggagagacggccaggatttcctgtggggaaaagagccttggaagtagagctgtacaatggtatcaacacagggccggccaggccccctctttaatcatatataataatcaggaccggccctcagggatccctgagcgattctctggctcccctgactccccttttgggaccacggccaccctgaccatcaccagtgtcgaagccggggatgaggccgactattactgtcatatatgggatagtagagttcccaccaaatgggtcttcggcggagggaccacgctgaccgtgttacgggctgatgctgcaccaactgtatccatcttctcaccatccagtgagcagttaacatctggaggtgcttcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcagatgcagttacaggagtcgggccccggactggtgaagccttcggaaaccctgtccctcacgtgcagtgtgtctggtgcctccataagtgacagttactggagctggatccggcggtccccagggaagggacttgagtggattgggtatgtccacaaaagcggcgacacaaattacagcccctccctcaagagtcgagtcaacttgtcgttagacacgtccaaaaatcaggtgtccctgagccttgtggccgcgaccgctgcggactcgggcaaatattattgcgcgagaacactgcacgggaggagaatttatggaatcgttgccttcaatgagtggttcacctacttctacatggacgtctggggcaatgggactcaggtcaccgtctcctcaggtaagctggcttttttctttctgcacattccattctgaaacgggatcgattgggaaataaactgtctagggatctcagagcctttaggacagattatctccacatctttgaaaaactaagaatctgtgtgatggtgttggtggagtccctggatgatgggatagggactttggaggctcatttgaagaagatgctaaaacaatcctatggctggagggatagttggggctgtagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggcctcgaggaat

SEQ ID NO: 35 - DNA, 2702 bp, homo sapiens
gcatagctagcgctcttcaaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgacagcgggacgcaagtagtgagggcactcagaacgccactcagccccgacaggcagggcacgaggaggcagctcctcaccctccctttctcttttgtcctgcgggtcctcagggagttagaacttgtttattgcagcttataatggttacaaataaagcaatagcatcacaaatttcacaaataaagcatttttttcactgcattctagttgtggtttgtccaaactcatcaatgtatcttatcatgtctggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagtgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatgggatggtcatgtatcatcctttttctagtagcaactgcaaccggtgtacattctgacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgagtttatcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgctctccaaagcggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtccatttgtcacagtctggggcagcggtgacgaagcccggggcctcagtgagagtctcctgcgaggcttccggatacaagattagtgaccactttattcattggtggcgacaggccccaggacagggccttcagtgggtggggtggatcaatcctaagactggtcagccaaacaatcctcgtcaatttcagggtagagtcagtctgactcgacaggcgtcgtgggactttgacacatattccttttacatggacctcaaggcagtaagatcggacgacacggccatttatttctgtgcgcgacaacgcagcgacttttgggatttcgacgtctggggcagcggcacgcaggtcactgtctcgtcaggtgagtcctcacaacctctctcctgctttaactctgaagggttttgctgctggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgactcgaggaatt

SEQ ID NO: 36 - DNA, 2744 bp, homo sapiens
gcatagctagcgctcttcaaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgacagcgggacgcaagtagtgagggcactcagaacgccactcagccccgacaggcagggcacgaggaggcagctcctcaccctccctttctcttttgtcctgcgggtcctcagggagttagaacttgtttattgcagcttataatggttacaaataaagcaatagcatcacaaatttcacaaataaagcatttttttcactgcattctagttgtggtttgtccaaactcatcaatgtatcttatcatgtctggtaacgagtggccaccttttcagtgttaccagtgagctctgagtgttcctaatgggaccaggatgggtctaggtgcctgctcaatgtcagagacagcaatggtcccacaaaaaacccaggtaatctttaggccaataaaatgtgggttcacagtgaggagtgcatcctggggttggggtttgttctgcagcgggaagagtgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgagagcatcacataacaaccacattcctcctctgaagaagcccctgggagcacagctcatcaccatgggatggtcatgtatcatcctttttctagtagcaactgcaaccggtgtacattcttcctatgtgcgcccgctgtcagtggccctgggggagacggccaggatttcctgtggacgacaggcccttggaagtagagctgttcagtggtatcaacataggccaggccaggcccctatattgctcatttataataatcaagaccggccctcagggatccctgagcgattctctggcacccctgatattaattttgggaccagggccaccctgaccatcagcggggtcgaagccggggatgaagccgactattactgtcacatgtgggatagtagaagtggcttcagttggtctttcggcggggcgaccaggctgaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgctctccaaagcggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaggcggaagcgggggtcaggagcaaccaacttttctctgctgaagcaagccggggacgtagaggaaaaccccggacccatgggatggtcatgtatcatcctttttctagtagcaactgcaaccggtgtacattctcaggtgcagctgcaggagtcgggcccaggactggtgaaaccttcggagaccctgtccgtcacctgcagtgtctctggagattccatgaataattactactggacttggatccggcagtcccccggaaagggactggagtggataggctatatctctgacagagaatcagcgacttacaacccctccctcaatagtcgagtcgtcatatcacgagacacgtcgaaaaaccaattgtccctaaaattaaactccgtcacccctgcggacacggccgtctattactgtgcgacagcgcgccgaggacagaggatttatggagtggtttcctttggagagttcttctactactactccatggacgtctggggcaaggggaccacggtcaccgtctcctcaggtgagtcctcacaacctctctcctgctttaactctgaagggttttgctgctggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgactcgaggaatt

SEQ ID NO: 37 - DNA, 20 bp, artificial sequence
  primer
cctggccccattgttcctta

SEQ ID NO: 38 - DNA, 20 bp, artificial sequence
  primer
gcgtctcaggacctttgtct

SEQ ID NO: 39 - DNA, 20 bp, artificial sequence
  primer
aatgtctgagttgcccaggg

SEQ ID NO: 40 - DNA, 20 bp, artificial sequence
  primer
tgtcacagaggtggtcctga

SEQ ID NO: 41 - DNA, 20 bp, artificial sequence
  primer
atggctgcaaagagctccaa

SEQ ID NO: 42 - DNA, 20 bp, artificial sequence
  primer
ggaaaaagggtcagaggcca

SEQ ID NO: 43 - DNA, 20 bp, artificial sequence
  primer
tgccctgtgattatccgcaa

SEQ ID NO: 44 - DNA, 20 bp, artificial sequence
  primer
gagctggaggaccgcaatag

SEQ ID NO: 45 - DNA, 20 bp, artificial sequence
  primer
gccactctagggcctttgtt

SEQ ID NO: 46 - DNA, 20 bp, artificial sequence
  primer
agcttcaaggcactgaggtc

SEQ ID NO: 47 - DNA, 19 bp, artificial sequence
  primer
ctacatggacgtctggggc

SEQ ID NO: 48 - DNA, 20 bp, artificial sequence
  primer
ctgctctcatcaagaccggg

SEQ ID NO: 49 - protein, 18 aa, artificial sequence
  self cleaving sequence in modified protein
EGRGSLLTCGDVEENPGP

SEQ ID NO: 50 - protein, 19 aa, artificial sequence
  self cleaving sequence in modified protein
ATNFSLLKQAGDVEENPGP

SEQ ID NO: 51 - protein, 20 aa, artificial sequence
  self cleaving sequence in modified protein
QCTNYALLKLAGDVESNPGP

SEQ ID NO: 52 - protein, 22 aa, artificial sequence
  self cleaving sequence in modified protein
VKQTLNFDLLKLAGDVESNPGP

(19) United States
(12) Patent Application Publication
(10) Pub. No.: US 2021/0024889 A1
(43) Pub. Date: Jan. 28, 2021

(54) ANTIBODY GENE EDITING IN B LYMPHOCYTES

(71) Applicant: The Rockefeller University, New York, NY (US)

(72) Inventors: Harald Hartweger, New York, NY (US); Mila Jankovic, New York, NY (US); Michel Nussenzweig, New York, NY (US)

(21) Appl. No.: 16/938,132

(22) Filed: Jul. 24, 2020

Related U.S. Application Data
(60) Provisional application No. 62/877,982, filed on Jul. 24, 2019.

(51) Int. Cl. C12N 5/0781; C12N 9/22; C07K 16/10; C12N 15/90

(52) U.S. Cl. CPC C12N 5/0635; C12N 9/22; C07K 16/1045; C12N 2510/02; C12N 2310/20; C07K 2317/76; C12N 15/907

(57) ABSTRACT

Provided are compositions and methods that relate to engineering B cells that express heterologous antibodies. The B cells are modified using CRISPR-based approaches. The modified B cells maintain allelic exclusion, and are produced such that endogenous Ig genes are silenced, such as by insertion of a bi-cistronic cDNA into the Igh locus. Functional antibodies are produced by expression of the bi-cistronic cDNA. The modified B cells can be engineered to produce antibodies to any particular epitope. The modified B cells may be administered to an individual who is subsequently vaccinated with a composition comprising the epitope to stimulate production of the antibodies.

### CROSS REFERENCE TO RELATED APPLICATIONS

This application claims priority to U.S. provisional patent application No. 62/877,982, filed Jul. 24, 2019, the entire disclosure of which is incorporated herein by reference.

### STATEMENT REGARDING FEDERALLY SPONSORED RESEARCH

This invention was made with government support under grant nos. 1UM1AI100663 and R01AI-129795 awarded by the National Institutes of Health. The government has certain rights in the invention.

### FIELD

The present disclosure relates to modified B cells and methods for making the modified B cells. The B cells maintain allelic exclusion and produce heterologous antibodies when introduced to an individual.

### BACKGROUND

Although a vaccine for HIV remains elusive, anti-HIV-1 broadly neutralizing-antibodies (bNAbs) have been identified and their protective activity has been demonstrated in animal models (Escolano et al., 2017; Kwong and Mascola, 2018; Nishimura and Martin, 2017; Sok and Burton, 2018). These antibodies are effective in suppressing viremia in humans and large-scale clinical trials to test their efficacy in prevention are currently under way (Bar et al., 2016; Caskey et al., 2015; Caskey et al., 2017; Ledgerwood et al., 2015; Lynch et al., 2015; Mendoza et al., 2018; Nishimura and Martin, 2017; Scheid et al., 2016; Schoofs et al., 2016). However, these antibodies typically have one or more unusual characteristics including high levels of somatic hypermutation (SHM), long or very short complementarity determining regions (CDRs) and self-reactivity that interfere with their elicitation by traditional immunization.

Consistent with their atypical structural features, antibodies that broadly neutralize HIV-1 have been elicited in camelids, cows and transgenic mice with unusual pre-existing antibody repertoires (Briney et al., 2016; Dosenovic et al., 2015; Escolano et al., 2016; McCoy et al., 2012; Sok et al., 2017; Tian et al., 2016). However, even in transgenic mice that carry super-physiologic frequencies of bNAb precursors, antibody maturation required multiple immunizations with a number of different sequential immunogens. Moreover, bNAbs only developed for one of the epitopes targeted (Briney et al., 2016; Escolano et al., 2016; Tian et al., 2016). Consequently, elicitation of bNAbs in primates or humans remains a significant challenge. There is accordingly and ongoing an unmet need for compositions and methods safe and effective for production of antibodies, including but not necessarily limited to antibodies with virus neutralizing activity. The present disclosure is pertinent to this need.

### SUMMARY

The present disclosure provides, in one embodiment, a method to produce transgenic antibodies in primary B cells using CRISPR-based systems. This new method involves short term culture in vitro, silencing of the endogenous Ig genes, and insertion of a bi-cistronic cDNA into the Igh locus. Mouse B cells edited to express an anti-HIV-1 bNAbs by this method can produce transgenic antibody levels that are protective in animal models (Mascola et al., 1999; Parren et al., 2001; Shibata et al., 1999; Shingai et al., 2014).

Mouse and human B lymphocytes typically express a single antibody despite having the potential to express 2 different heavy chains and 4 different light chains. Theoretically the combination could produce 8 different antibodies and a series of additional chimeras that could interfere with the efficiency of humoral immunity and lead to unwanted autoimmunity. Allelic exclusion prevents this from happening and would need to be maintained by any gene replacement strategy used to edit B lymphocytes. In addition, genetic editing is accompanied by safety concerns due to off-target double strand breaks and integrations. The currently provided approach lowers these risks by using non-viral gene editing with ssDNA templates, which limits random integrations and by keeping culture time short to prevent expansion of any such cell.

The present approach maintains allelic exclusion in part by ablating the Igkc gene. In the mouse data described below, 95% of B cells express Igkc. In the absence of Igkc expression these cells will die by apoptosis because they cannot survive unless they continue to express a B cell receptor (Kraus et al., 2004; Lam et al., 1997). Since the introduction of the transgene into the heavy chain locus disrupts endogenous Igh expression, editing maintains allelic exclusion in the majority of cells because only cells expressing the introduced antibody can survive. The presently provided strategy also interferes with the survival of cells that suffer off-target integration events, because the majority of such cells would be unable to express the B cell receptor and they too would die by apoptosis.

A potential issue is that there are two heavy chain alleles in every B cell and allelic exclusion would be disrupted if the transgene were only integrated in the non-productive Igh allele allowing for expression of the original productive Igh. However, our flow cytometry data discussed below indicates that this is a very rare event. Thus, either both alleles are targeted or the occasional remaining endogenous Igh gene is unable to pair with the transgenic Igk. A small number of B cells that have not deleted endogenous Igk might also integrate the transgene into the Igh locus. This could decrease the efficiency of knock-in antibody expression if the endogenous kappa pairs with the transgenic heavy chain. The use of a promoterless construct as described below increases surface BCR expression and improves safety. This construct relies on integration into an allele with in frame VDJ rearrangement. Furthermore, the absence of a promoter makes off target gene activation less likely thereby increasing the safety of this approach.

In contrast to the mouse, IGL is expressed by 45% of all B cells in humans. Therefore, this locus would either need to be ablated, or alternatively, cells expressing IGL could be removed from the transferred population by any one of a number of methods of negative selection. The disclosure includes each of these approaches.

Similar to antibody transgenes in mice, expression of the edited BCR varied between different antibodies. Some combinations of heavy and light chains were refractory to expression in mature B cells. In addition, although the level of B cell receptor expression was within the normal range, it was generally in the low end compared to polyclonal B cells. This is consistent with generally lower level expression of a similar transgene in knock-in mice (Jacobsen et al., 2018). Low BCR expression could also be due to the bi-cistronic design since expression was higher in knock-in mice that expressed the identical Ig from the native Igk and Igh loci (Dosenovic et al., 2018). Nevertheless, expression levels were adequate to drive antigen-induced antibody production in vivo.

bNAb mediated protection against infection with simian-human immunodeficiency viruses in macaques requires ICso neutralizing titers of 1:100 (Mascola et al., 1999; Parren et al., 2001; Shibata et al., 1999; Shingai et al., 2014). Thus, the titers achieved by CRISPR/Cas9 edited B cells in mice would be protective if they could be translated to macaques and by inference humans. Moreover, our neutralization measurements may be an underestimate since we excluded bNAbs produced as IgM or isotypes other than IgG.

Most protective vaccine responses depend on humoral immunity. Neutralizing antibody responses are readily elicited for most human pathogens, but in some cases, including HIV-1, it has not yet been possible to do so. The alternatives include passive antibody infusion, which has been an effective means of protection since it was discovered at the turn of the last century. However, in the present disclosure, it is shown that passive transfer of mouse B cells edited by CRISPR/Cas9 can also produce protective antibody levels in vivo. This demonstrates that humoral immune responses can be engineered by CRISPR/Cas9. The approach is not limited to HIV-1 and can be applied to any disease requiring a specific antibody response.

A non-limiting embodiment of the disclosure is illustrated by engineering mature B cells that express an anti-HIV-1 bNAb. Adoptive transfer of the engineered B cells and immunization with a single cognate antigen led to germinal center formation and antibody production at levels consistent with protection.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Efficient generation of indels in primary mouse B cells by CRISPR/Cas9. (A) Targeting scheme for Igh (crIgH) and Igk crRNA guides (crIgK₁, crIgK₂). (B) Experimental set up for (C-E). Primary mouse B cells were cultured for 24 h in the presence of anti-RP105 antibody and then transfected with Cas9 ribonucleoproteins (RNPs) and analyzed at the indicated time points. (C) Flow cytometric plots of cultured B cells at the indicated time points after transfection. Control uses an irrelevant crRNA targeting the HPRT gene. (D) Quantification of (C), percentage of Igκ⁻Igλ⁻ B cells by flow cytometry (right y-axis) and percentage of cells containing indels in the Igkc exon by TIDE analysis (left y-axis). Control bars include irrelevant HPRT-targeting crRNAs or a scramble crRNA without known targets in the mouse genome. (E) Percentage of cells containing indels in the J_{H}4 intron by TIDE analysis after targeting with crIgH or control. Bars indicate mean±SEM in two (TIDE) or four (flow cytometry) independent experiments.

FIG. 2. Engineering bNAb-expressing, primary, mouse B cells. (A) Schematic representation of the targeting strategy to create bNAb-expressing, primary mouse B cells. ssDNA homology-directed repair template (HDRT) contained 110 nt 5′ and 790 nt 3′ homology arms flanking an expression cassette. The 5′ homology arm is followed by the 111 nt long splice acceptor site and the first 2 codons of Cμ exon 1, a stop codon and a SV40 polyadenylation signal (CμSA SV40 pA). Then the mouse Ighv4-9 gene promoter, the leader, variable and joining regions (VJ) of the respective antibody light chain and mouse κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 45 nt of the mouse J_{H}1 intron splice donor site to splice into downstream constant regions. (B) Experimental setup for (C). (C) Flow cytometric plots of primary, mouse B cells, activated and transfected with RNPs targeting the Ighj4 intron and Igkc exon with or without ssDNA HDRTs encoding the 3BNC60^{SI}, 3BNC117 or 10-1074 antibody. Non-transfected, antigen-binding B cells from 3BNC60^{SI} knock-in mice cultured the same way are used as control for gating. (D) Quantification of (C). Each dot represents one transfection. Data from 7 independent experiments (B-D). (E) Experimental set up for (F-H) (F) Flow cytometric plots of primary, mouse B cells, activated and transfected using ssDNA HDRT encoding the antibodies 3BNC60^{SI}, 3BNC117, PGT121 or 10-1074. B cells were expanded on feeder cells for 3 days. Cultured, non-transfected, antigen-binding B cells from PGT121 knock-in mice are shown for gating. (G) Quantification of (F). (H) Total number of antigen binding B cells before (24 h) or after 3 days (day 4) of feeder culture. Bars indicate mean±SEM. Combined data from 2 independent experiments for (E-H).

FIG. 3. Engineering bNAb-expressing, primary, human B cells. (A) Schematic representation of the targeting strategy to create bNAb-expressing, primary human B cells. The ssDNA HDRT is flanked by 179 nt and a 521 nt homology arms. The central expression cassette contains 112 nt of the human splice acceptor site and the first 2 codons of Cμ exon 1, a stop codon and a SV40 polyadenylation signal (CμSA SV40 pA). Then the human IGHV1-69 gene promoter, the leader, variable and joining regions (VJ) of the respective antibody light chain and human κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 50 nt of the human J_{H}4 intron splice donor site to splice into downstream constant regions. (B) Experimental set up for (C, D). Primary human B cells were cultured for 24 h in the presence of anti-RP105 antibody and then transfected with RNPs±HDRT. (C) Flow cytometric plots of primary human B cells 48 h after transfection with RNPs containing crRNAs without target (scramble) or targeting the IGHJ6 intron or the IGKC exon. (D) Quantification of (C). Bars indicate mean±SEM. Combined data from 3 independent experiments is shown (B-D). (E) Flow cytometric plots of antigen binding by Igλ⁻ primary human B cells 72 h after transfection of RNPs targeting both the IGHJ6 intron and the IGKC exon with or without HDRTs encoding 3BNC60^{SI} or 10-1074. (F) Quantification of (E). Bars indicate mean±SEM. Combined data from 2 independent experiments with 2-4 replicates each (E, F).

FIG. 4. Engineered bNAb-expressing primary mouse B cells participate in humoral immune responses in vivo. (A) Experimental setup for B-E. (B) Anti-3BNC60^{SI} idiotype-coated, mouse IgG ELISA of sera from mice adoptively transferred with the indicated B cells and immunized with the cognate antigen TM4 core at the indicated time points. Representative plots of seven independent experiments. (C) Anti-3BNC60^{SI} idiotype-coated mouse IgG1a or IgG1b ELISA of day 14 sera, as above. Representative plots of two independent experiments. (D) 3BNC60^{SI} serum IgG levels 14 d after immunization in mice transferred with 3BNC60SI-edited cells. Numbers of total B cells/mouse at transfection are indicated. Cells were transferred either 24 h after transfection or after additional culture on feeder cells as in FIG. 2 D. Determined by anti-3BNC60^{SI} idiotype-coated mouse IgG ELISA over seven independent experiments. Each dot represents one mouse, and the line indicates the arithmetic mean. (E) TZM.b1 neutralization data of protein G-purified serum immunoglobulin days 14-21 after immunization from mice treated as in A but transfected with 10-1074 HDRT and immunized with cognate antigen 10mut. Combined data from two independent experiments are shown.

FIG. 5. Cultured B cells participate in humoral immune responses. (A) Schematic representation of the experimental set up for (B) and (C). B1-8^{hi} CD45.1 Igh^{a} cells were cultured for 24 or 48 h in the presence of anti-RP105 antibody, then rested for 2-3 h without antibody and then transferred into C57BL6/J (CD45.2 Igh^{b}) recipients. 18 h later, mice were immunized with NP-OVA i.p. and mice were analyzed 2 weeks later. (B) Flow cytometric plots gated on CD38⁻Fas⁺GL7⁺IgD⁻ GC B cells 2 weeks after transfer. (C) Pre-immune (day 0) and day 13 ELISA titers of anti-NP IgG1^{a} or IgG1^{b}. (D) Schematic representation of the experimental set up for (E). B1-8^{hi} CD45.1 Igh^{a} cells were cultured for 24 h and transfected with plasmid DNA. 24 h after transfection cells were transferred and analyzed as in (A). (E) Flow cytometric plots gated on CD38⁻Fas⁺GL7⁺IgD⁻ GC B cells 11 days after transfer. Data (A-E) are representative of 2-3 independent experiments.

FIG. 6. Identification of optimal mouse Igh crRNA and ssDNA HDRT template production. (A) Schematic representation of the mouse Igh locus around J_{H}4. Location and sequence of tested guide RNAs is indicated below. (B) TIDE assay comparing the efficiency of creating indels of the crRNAs indicated in (A). Forward/reverse indicate sequencing with forward/reverse primers respectively. Representative of 2 independent experiments. (C) Flow chart of ssDNA production. HDRT templates were cloned into pLSODN-4D, Maxi-prepped, sequence verified and digested with restriction enzyme Xhol and the nicking endonuclease Nt.BspQI to produce 3 ssDNA fragments of the vector. Denaturing loading buffer was used to separate the 3 fragments by conventional agarose gel electrophoresis as indicated. ssDNA HDRT quality and integrity was verified using (D) Bioanalyzer and (E) agarose gel electrophoresis. Representative of >20 independent preparations.

FIG. 7. Cell viability and Igh allelic exclusion of bNAb expressing murine B cells. (A) Flow cytometric plots showing percentage of live cells among all events 48 h after RNP±HDRT transfection. Related to FIG. 2 B, C. (B) Experimental set up for (C). Heterozygous (Igh^{a/b}) B cells expressing IgH^{a} or IgH^{b} alleles were activated for 24 h, then transfected with 3BNC60^{SI} HDRT and analysed 48 h later. (C) Overlays of flow cytometric plots of TM4 core binding cells and non-binding B cells, both pre-gated on λ⁻ B cells. TM4 core mean fluorescence intensity (5.89×10³ to 1.28×10⁵) is color mapped onto TM4 core-binding cell population. Numbers represent the percentage of TM4 core-binding cells among λ⁻ B cells (left) or the percentage of TM4 core-binding B cells in the respective gate (right). Concatenate of 5 technical repeats in 2 independent experiments is shown (B-C). (D) Schematic representation of the promoterless targeting strategy to create bNAb-expressing, primary mouse B cells. ssDNA homology-directed repair template (HDRT) contained 110 nt 5′ and 790 nt 3′ homology arms flanking an expression cassette. The 5′ homology arm is followed by the 111 nt long splice acceptor site and the first 2 nucleotides of Cμ exon 1 and an in-frame T2A sequence with GSG linker. Then the leader, variable and joining regions (VJ) of the respective antibody light chain and mouse κ constant region (C_{κ}) are followed by a furin-cleavage site, a GSG-linker and a P2A self-cleaving oligopeptide sequence (P2A), the leader, variable, diversity and joining regions (VDJ) of the respective antibody heavy chain and 45 nt of the mouse J_{H}1 intron splice donor site to splice into downstream constant regions. (E) Flow cytometry of mouse B cells transfected and analysed as in FIG. 2 B either without template, or promoter-driven template or promoterless HDRT encoding 3BNC60^{SI}. Left panel shows cognate antigen binding (TM4 core) and right panel identifies correctly edited B cells using anti-idiotypic antibody iv8. (F) Geometric mean fluorescence intensity of TM4 core-binding of cells gated as in the left panel of (E). Bars indicate mean±SEM. Representative of 2 independent experiments.

FIG. 8. TIDE analysis and viability of primary, human B cells after transfection. (A) TIDE assay 42 h after transfection, comparing the efficiency of creating indels of crRNAs targeting the human IGKC exon and (B) TIDE assay using 2 different primer sets, 24 h after transfection, comparing the efficiency of creating indels of crRNAs targeting the human IGHJ6 intron. Forward/reverse indicate sequencing with forward/reverse primers respectively. Representative of 2 independent experiments. (C) Flow cytometric plots showing percentage of live cells among all events 72 h after RNP±HDRT transfection. Related to FIG. 4 D. Representative plots of 2 independent experiments are shown.

FIG. 9. Serum neutralization of wild type mice adoptively transferred with edited B cells. Related to FIG. 4. (A, B) Neutralization curves for HIV strains T240-4 (A) and Q23.17 (B) of data summarized in FIG. 4 E of mice receiving 10-1074-edited B cells and immunized with cognate antigen 10mut. (C) HIV neutralization data of mice receiving 3BNC60^{SI}-edited B cells and immunized with cognate antigen TM4 core. Combined data from 2 independent experiments (A-C).

FIG. 10. Color annotated version of Table 2.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. All time intervals, temperatures, reagents, culture conditions and media, described herein are included in this disclosure.

The disclosure includes all steps and compositions of matter described herein in the text and figures of this disclosure, including all such steps individually and in all combinations thereof, and includes all compositions of matter including but not necessarily limited to vectors, cloning intermediates, cells, cell cultures, progeny of the cells, and the like. The disclosure includes all polynucleotide sequences, their RNA or DNA equivalents, all complementary sequences, and all reverse complementary sequences. If reference to a database entry is made for a sequence, the sequence is incorporated herein by reference as it exists in the database as of the filing date of this application or patent. Sequences that are 80.0-99.9% identical, inclusive, and including all numbers to the first decimal point there between, to any nucleotide or amino acid sequence are encompassed by this disclosure. The disclosure includes the human homologues of every mouse nucleotide and amino acid sequence described herein.

All nucleotide sequences and amino acid sequences encoded by them are included in this disclosure. The disclosure includes contiguous segments of these sequences. The disclosure includes all sequences encoding leader, variable, and joining regions (VJ) encoding antibody light chains, and all the sequences encoding the variable, diversity, and joining regions (VDJ) of heavy chains, and all amino acid sequences encoded by those sequences. CDR sequences from these sequences can be recognized by those skilled in the art and are also included as distinct sequences. The disclosure includes all combinations of VJ and VDJ sequences, combinations of distinct antibodies that are differentiated from one another by said sequences, modified B cells that encode such antibodies, and combinations of modified B cells that produce distinct antibodies, as further described herein.

Throughout this application, unless stated differently, the singular form encompasses the plural and vice versa. All sections of this application, including any supplementary sections or figures, are fully a part of this application.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. In embodiments, about 3×10⁴-4×10⁵ modified B cells/kg are administered, such as by intravenous administration.

This disclosure provides modified B cells, antibodies made by such B cells, vectors and cells comprising nucleic acids encoding the antibodies, compositions comprising any of the foregoing, methods of making any of the foregoing, and methods of using the modified B cells expressing the antibodies in the treatment and/or prophylaxis of a condition associated with the antigen to which the produced antibodies bind with specificity. This disclosure includes all nucleic acid and amino acid sequences described herein and all contiguous segments thereof including all integers and ranges of integers there between. In embodiments, each CDR amino acid sequence of each antibody of this disclosure is included as a distinct sequence.

In an embodiment, the disclosure provides a method for modifying one or more primary B cells to provide one or more modified primary B cells. The modified primary B cells maintain allelic exclusion and can participate in a humoral immune response when introduced into a mammal. The modified primary B cells can also form germinal centers in the individual into which they are introduced. The modified primary B cells produce heterologous antibodies that bind with specificity to a distinct epitope. “Heterologous” means the modified B cells produce antibodies that are encoded by the constructs described herein and which are introduced into the modified B cells. Thus, in embodiments, the antibodies are not encoded by the primary B cells before being modified as set forth in this disclosure. Primary B cells are B lymphocytes that are characterized by having developed in in vivo. In embodiments, primary mature naïve B cells derived from blood or spleen are used. In embodiments, the primary B cells may be memory B cells. In embodiments, the primary B cells used in the methods of this disclosure are IgM or IgD and do not detectably express activation markers at the time they are modified. Memory B cells can express IgM or IgD, or any of switched isotypes. In human samples, CD27 may be used to indicate memory B cells. In embodiments, the disclosure comprises obtaining B cells from an individual, modifying the B cells as described herein, and administering the B cells to the individual. As described further herein, production of the heterologous antibodies can be stimulated by vaccinating the individual with the epitope to which the heterologous antibodies bind with specificity.

In embodiments, the antibodies produced by modified B cells of this disclosure produce functional antibodies. “Functional antibodies” are antibodies that bind to their cognate epitope with specificity. Thus, modified B cells of this disclosure express the sequence encoding leader, variable, and joining regions (VJ) of the heterologous antibody light chain, and the sequence encoding the variable, diversity, and joining regions (VDJ) of the heterologous heavy antibody chain, to thereby form a functional antibody.

The antibodies produced by the modified B cells can contain any suitable framework and hypervariable regions. Any desired complementarity determining regions (CDRs) can be part of the antibodies produced by the modified B cells. Aspects of the disclosure are illustrated with modified B cells that produce IgG and/or IgM antibodies, but the method can be adapted, given the benefit of this specification, to produce any isotope (e.g., any of IgA, IgD, IgE, IgM, and IgG).

The epitope to which the antibodies produced by modified B cells bind is not particularly limited. In embodiments, the epitope (and thus the CDRs of the antibodies produced by the modified B cells confer specificity to the epitope) is present on any infectious agent, such as an infectious microorganism, or a virus. In embodiments, the infectious organism is any pathogenic bacteria, or an infectious pathogenic fungus. In embodiments, the epitope is present on the surface of a virus, or on a component of a virus that is exposed during replication or during cell entry.

In embodiments, the epitope is present on a virus that specifically infects humans, or specifically infects non-human animals, or avian animals. The disclosure thus includes human and veterinary approaches.

In embodiments, the modified B cells produce viral neutralizing antibodies. The term “neutralizing antibody” and its various grammatical forms refers to antibodies that inhibit, reduce or completely prevent viral infection. Whether neutralizing antibodies are produced can be determined by in vitro assays that are known in the art. Modified B cells of this disclosure can be used for prophylaxis and/or treatment of, for example, viral infections. In embodiments, the antibodies bind with specificity to an epitope comprised by any component of HIV, or any component of a coronavirus, or any component of a hepatitis virus.

In non-limiting embodiments, antibodies produced by modified B cells of this disclosure are specific for any epitope include anti-HIV antibodies PGT121, 3BNC60^{SI}, 10-1074, and 3BNC117, and variants and derivatives thereof. In non-limiting embodiments, the antibodies produced by the modified B cells of this disclosure are any antibody, and variants and derivatives thereof, as described in PCT publication WO/2018/187799, published Apr. 9, 2018, the entire disclosure of which is incorporated herein by reference.

In embodiments, a CRISPR system is used to initially introduce a ssDNA homology directed repair template (HDRT) into primary B cells. Insertion of the HDRT may be heterozygous or homozygous for any particular allele.

The HDRT comprises or consists of at least the following elements:
a) first homology arm;
b) a splice acceptor site;
c) nucleotides from constant mu (Cμ) exon 1;
d) a sequence encoding a first amino acid linker sequence;
e) a sequence encoding a first self-cleaving amino acid sequence;
f) a sequence encoding leader, variable, and joining regions (VJ) of the heterologous antibody light chain;
g) a sequence encoding a kappa constant region (C_{κ});
h) a sequence encoding a protease-cleavage site;
i) a sequence encoding a second amino acid linker sequence;
j) a sequence encoding a second self-cleaving amino acid sequence;
k) a sequence encoding leader, variable, diversity, and joining regions (VDJ) of the heterologous heavy antibody chain;
l ) an intron splice donor site;
m) a second homology arm.

In embodiments, the splice acceptor may comprise an AG nucleotide sequence, and may further comprise a branch sequence. In embodiments, nucleotides from constant mu (Cμ) exon 1 are from any suitable such exon sequence. In embodiments, the nucleotides are inserted such that they maintain the downstream reading frame of the remainder of the construct, and any number of nucleotides can be used. Since the first codon in the exon is split between the J and the constant region with the first nucleotide encoded by J and the other two nucleotides by the C region, the following equation applies for the number of nucleotides (nucleotides x 3)-1. The sequence of the constant mu (Cμ) exon 1 is known and can be accessed at, for example, NCBI Gene ID 3507, Ensemb1 ENSG00000211899.

The first amino acid linker is typically three amino acids long, and may be comprised of a GSG sequence. The first self-cleaving amino acid sequence is typically about 18-22 amino acids long. Any suitable sequence can be used, non-limiting examples of which include: T2A, comprising the amino acid sequence: EGRGSLLTCGDVEENPGP (SEQ ID NO:49); P2A, comprising the amino acid sequence ATNFSLLKQAGDVEENPGP (SEQ ID NO:50); E2A, comprising the amino acid sequence QCTNYALLKLAGDVESNPGP (SEQ ID NO:51); and F2A, comprising the amino acid sequence VKQTLNFDLLKLAGDVESNPGP (SEQ ID NO:52).

The kappa constant region is known, and can be accessed at, for example NCBI Gene ID 3514, Ensembl ENSG00000211592. Alternatively, a lambda constant region may be used, and its sequence is also know. The sequence of the protease-cleavage site is typically about 4 amino acids long. In a non-limiting embodiment, the sequence is RRKR. The sequence of the second amino acid linker sequence may be the same as the first amino acid linker. The intron splice donor site can be of variable length, typically about 40-60 nucleotides. In embodiments, the intron splice donor comprises a GU sequence.

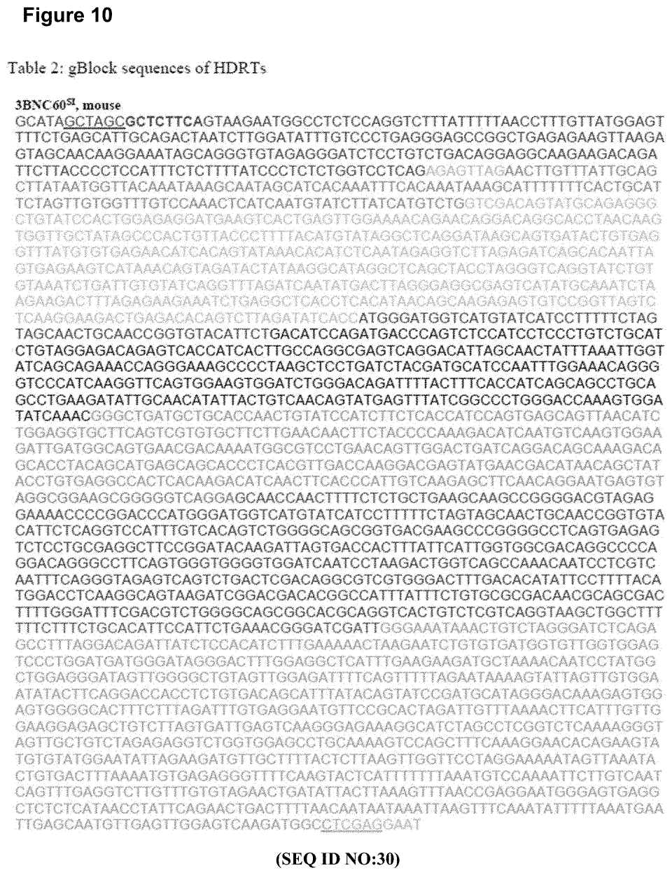

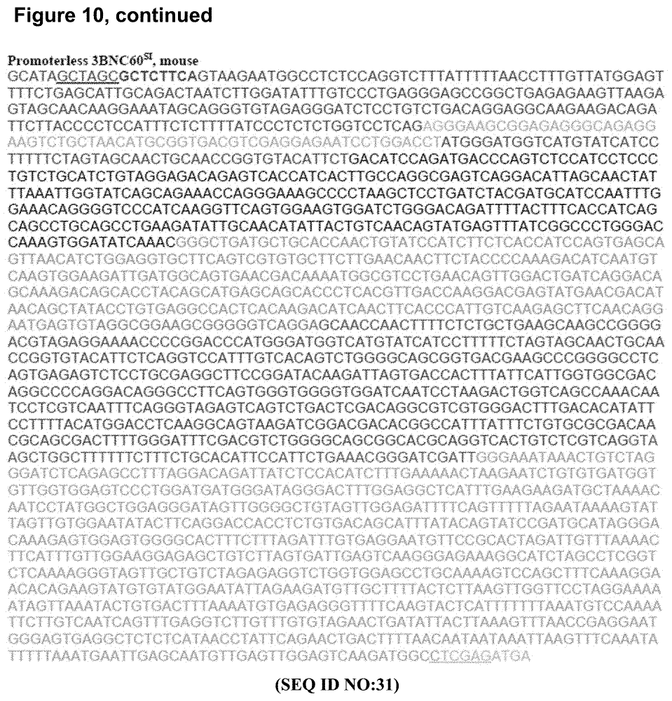

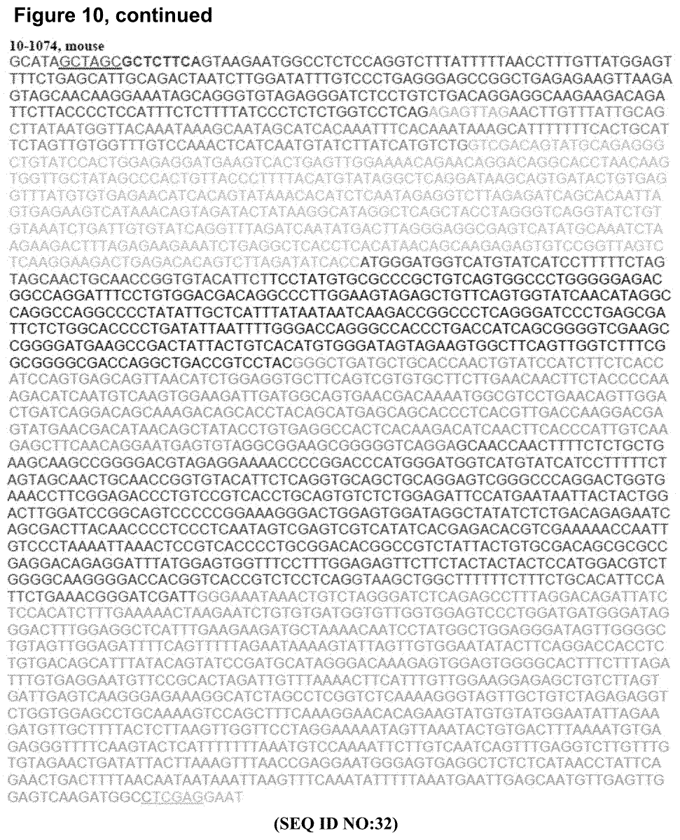

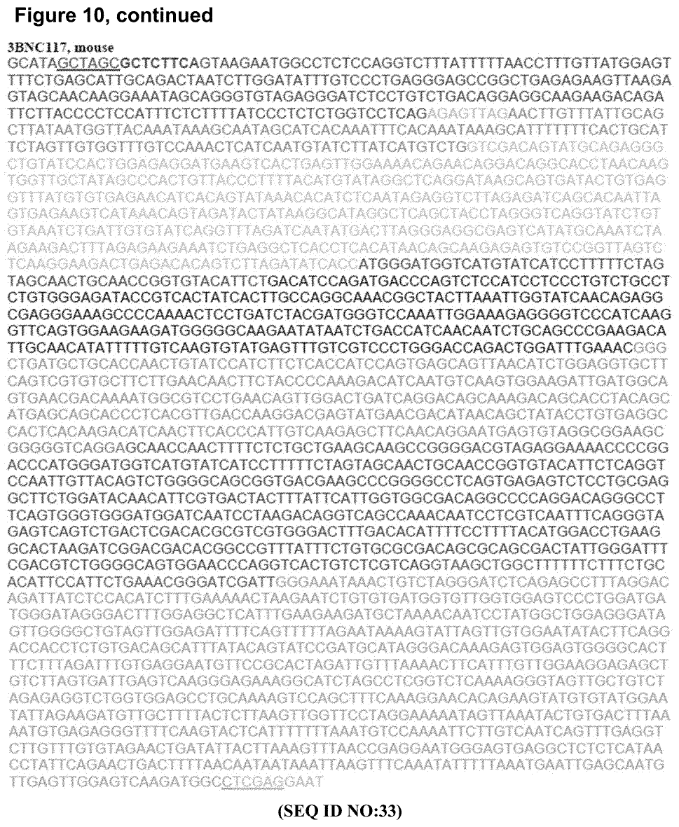

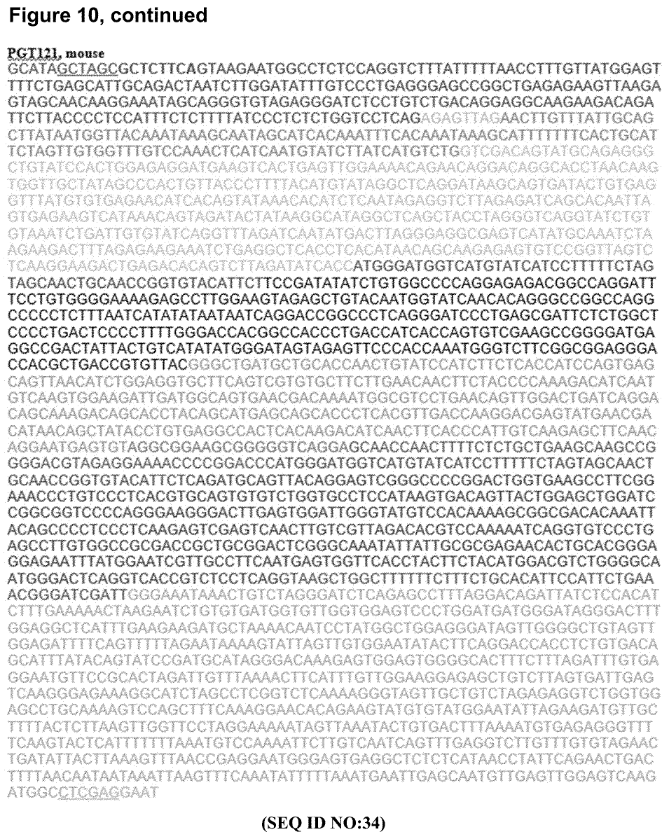

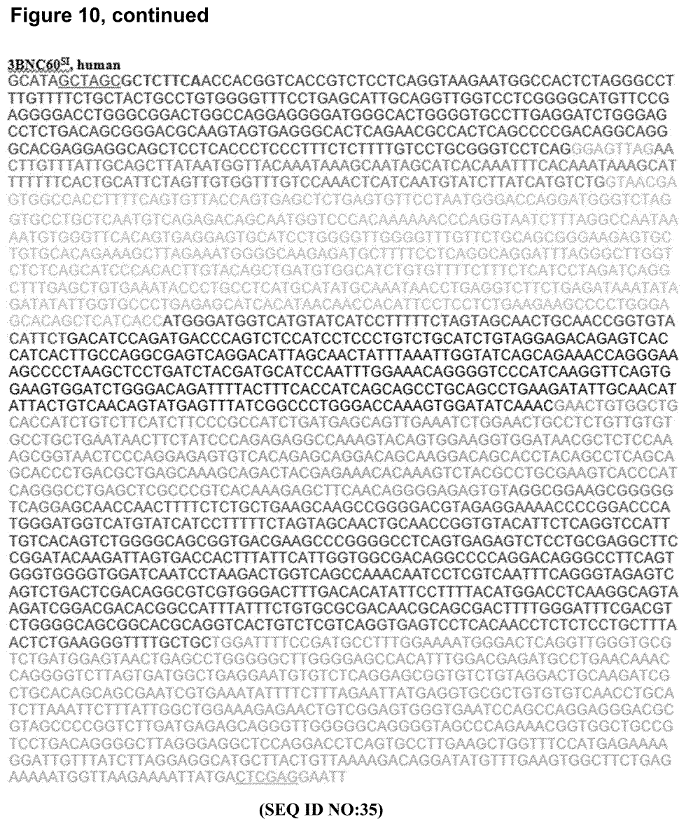

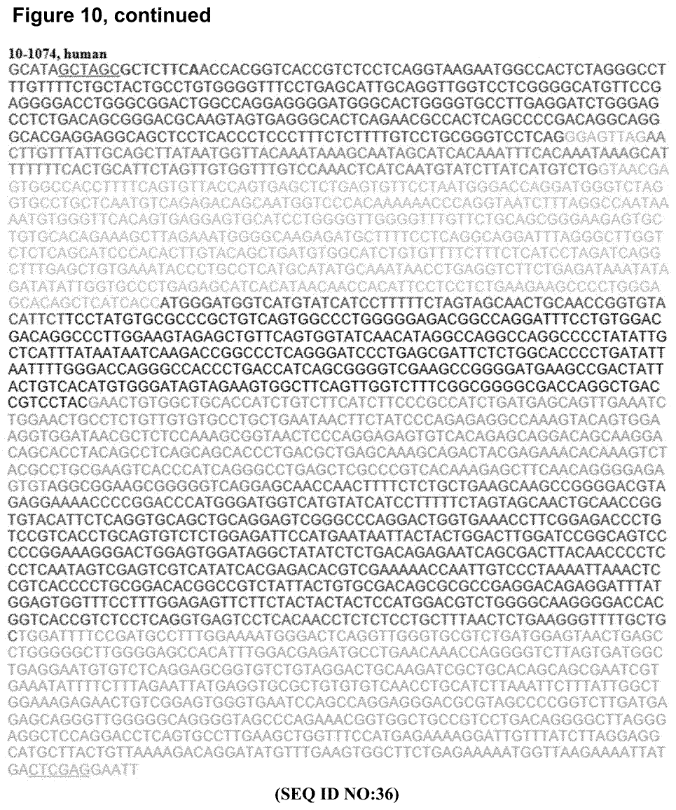

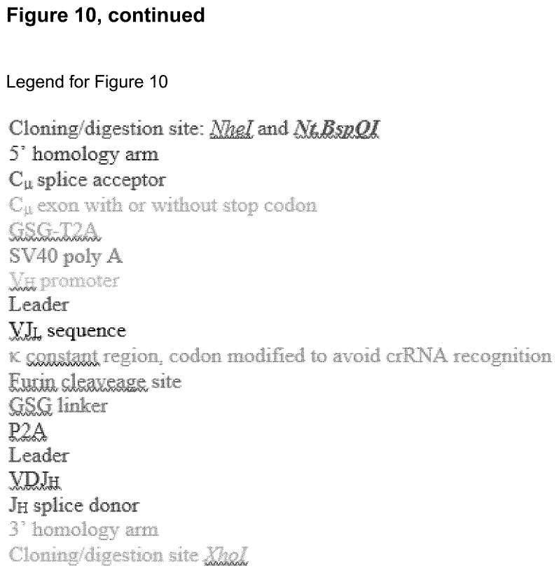

The first and second homology arms are configured to be introduced into one or more desired chromosomal loci. In embodiments, the disclosure comprises combined endogenous Ig disruption with insertion of a transcription unit (the HDRT) that directs expression of the heavy and light chain into an endogenous heavy chain locus/loci. In embodiments, such loci comprise the IGKC exon, an IGHJ6 intron, an IgLC locus, or a combination thereof. The sequence of the IGKC exon can be accesed at, for example, NCBI Gene ID 3514, Ensembl ENSG00000211592). The sequence of IGHJ6 introns can be accessed at, for example, NCBI Gene ID 28475, Ensembl ENSG00000211900). There are five functional genes in the IgLC locus, and any can be used for the homology arm. The sequence of the five genes in the IgLC locus are IGLC1, IGLC2, IGLC3, IGLC6 IGLC7, and can be accessed at, for example, NCBI Gene ID 3537, 3538, 3539 3542, 28834, respectively, Ensemb1 ENSG00000211675, ENSG00000211677, ENSG00000211679, ENSG00000222037, ENSG00000211685, respectively. Thus, the sequences of the first and second homology arms may be identical to the chromosomal sequences into which they are introduced and/or replace. Non-limiting examples of HDRT sequences used in this disclosure are provided in Table 2 and FIG. 10. In embodiments, the homology arms are from 60 nucleotides to about 3 Kb in length.

In addition to the HDRT, the disclosure comprises introducing into primary B cells a clustered regularly interspaced short palindromic repeats (CRISPR)-Cas (CRISPR-associated proteins) system. The disclosure is illustrated using a Cas9 enzyme, but it is expected that other CRISPR systems and Cas enzymes can be used. In embodiments, any type II CRISPR system/Cas enzyme is used. In embodiments, the type II system/Cas enzyme is type II-B enzyme. In embodiments, that Cas enzyme comprises Cpf1

In embodiments, the disclosure includes introducing the HDRT, the Cas enzyme, a trans-activating crRNA (tracrRNA), and one, two, or three guide RNAs. Suitable tracrRNAs are known in the art and can be adapted for use with the methods of this disclosure. The guide RNAs may be provided as crRNAs. The guide RNAs are programmed to target specific sites so that the first and second homology arms are integrated correctly, depending on the locus where the HDRT is inserted. In embodiments, two or three guide RNAs are used. In embodiments, the guide RNAs are targeted to a suitable sequence in the IGKC, IGHJ6, and/or IgLC loci.

Methods for designing suitable guide RNAs are known in the art such that guide RNAs having the proper sequences can be designed and used, when given the benefit of the present disclosure. Non-limiting examples of guide RNAs are provided in Table 1.

In embodiments, insertion of an HDRT described herein into a plurality of primary B cells results in more of the primary B being λ-B cell receptor positive primary B cells than κ-B cell receptor positive primary B cells. In embodiments, insertion of an HDRT as describe herein reduces or eliminates λ-B cell receptor positive primary B cells, and/or reduces or eliminates κ-B cell receptor positive primary B cells.

In embodiments, an HDRT of this disclosure comprises at least one of the following characteristics:

i) no promoter is included in the HDRT;

ii) the primary B cells are human B cells;

iii) only two nucleotides from the Cμ exon 1 are included in the HDRT;

iv) the first or second self-cleaving amino acid sequences comprise a T2A sequence or a P2A sequence;

v) the first or second amino acid linker sequences, or both, are GSG-linker sequences;

vi) the protease cleavage site is a furin-cleavage site;

vii) the CAS enzyme and the guide RNAs are introduced into the primary B cell as a ribonucleotide protein complex;

vii) production of the modified primary B cells is performed without using a viral delivery vector.

In embodiments, the disclosure comprises providing a treatment to an individual in need thereof by introducing a therapeutically effective amount of modified B cells as described herein to the individual, and vaccinating the individual with an antigen that is cognate to the antibodies produced by the modified B cells. Thus, the antigen used in the vaccination comprises an epitope that is specifically recognized by the antibodies produced by the modified B cells. One or more vaccinations can be used.

In embodiments, the disclosure includes modified B cells made according to a method of this disclosure. In embodiments, the modified B cells can be provided in a pharmaceutical formulation, and such formulations are included in the disclosure. A pharmaceutical formulation can be prepared by mixing the modified B cells with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, Pa. Lippincott Williams & Wilkins, the disclosure of which is incorporated herein by reference.

In embodiments, the disclosure comprises a kit for use in making modified B cells. In embodiments, the kit can comprising one or more cloning vectors, the vectors comprising the elements discussed above for producing an HDRT, with the exception that the vector contains suitable restriction enzyme recognition sites for inserting a sequence encoding the VJ regions of the heterologous antibody light chain, and for inserting a sequence encoding the VDJ regions of the heterologous heavy antibody chain. Guide RNAs and a Cas enzyme may also be provided with the kit.

In embodiments, the disclosure comprises an isolated HDRT. Methods for producing ssDNA homology directed repair templates are known and can be adapted for use with the present disclosure. In a non-limiting embodiment, plasmids comprising the HDRT are digested with sequence-specific nickases, and ssDNA purification is performed using any suitable technique, such as by agarose gel electrophoresis.

In embodiments, the disclosure comprises isolating a sample from a mammal, identifying antibody coding sequences from the sample, and engineering B cells to express the identified antibody sequences. In embodiments, the individual produces virus neutralizing antibodies, and the engineered B cells make antibodies that neutralize the same virus, e.g., the produced antibodies comprise the same CDRs as the antibodies in the sample.

In embodiments, the disclosure comprises obtaining a sample comprising B cells from an individual, determining the sequence of the VJ regions of an antibody light chain, determining the sequence of the VDJ chain of the same antibody, and generating an HDRT comprising the sequences encoding the VJ and VDJ regions. The disclosure further includes using the HDRT to produce modified B cells that comprise the VJ and VDJ regions, and which produce the antibodies.

The following examples are meant to illustrate, and are not intended to be limiting. The disclosure includes all reagents and process steps that are described in these examples.

### EXAMPLE 1

Expressing Antibodies in Primary Mature, Murine B Cells.

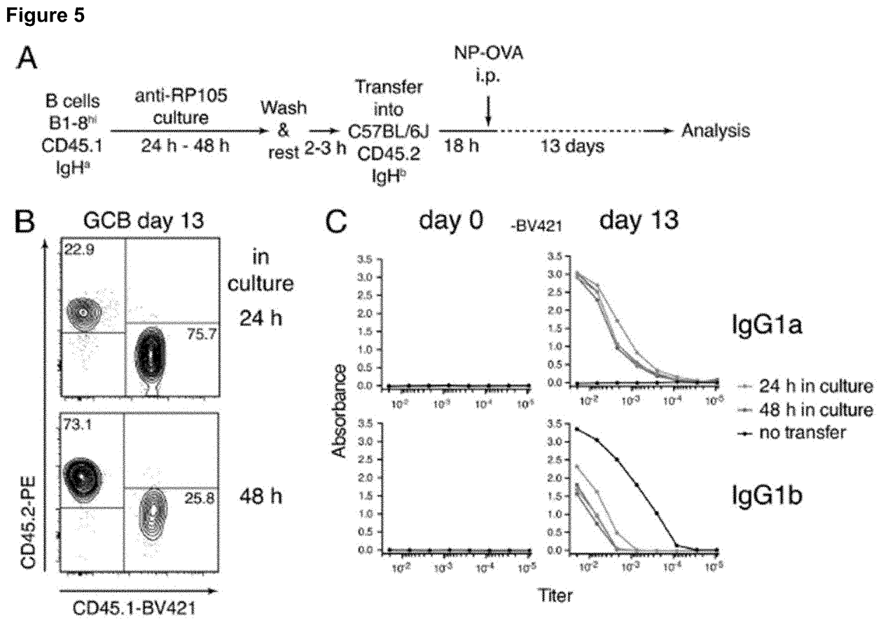

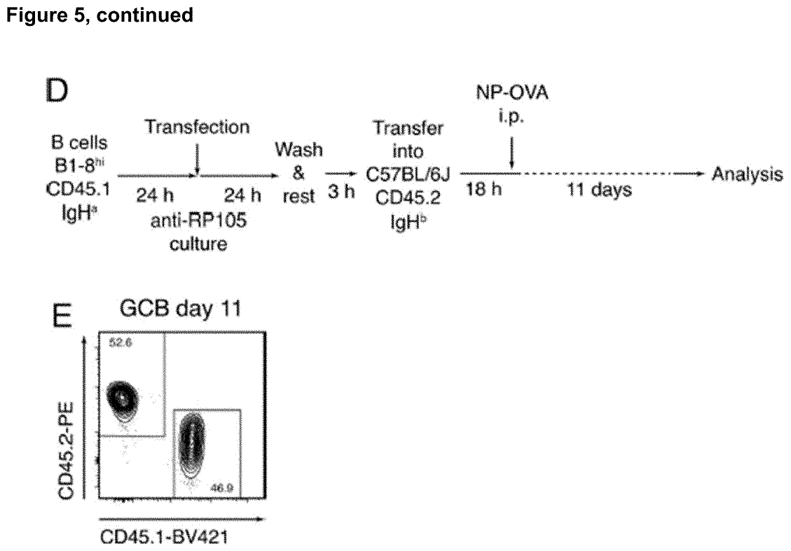

To edit mature B cells efficiently, the disclosure comprise activating and culturing the B cells in vitro. To determine whether such cells can participate in humoral immune responses in vivo we used Igh^{a} CD45.1 B cells carrying the B1-8^{hi} heavy chain that are specific for the hapten 4-hydroxy-3-nitro-phenylacetyl (NP) (Shih et al., 2002). B1-8^{hi} B cells were activated in vitro with anti-RP105 antibody for 1-2 days and subsequently transferred into congenically marked (Igh^{b} CD45.2) C57BL/6J mice. Recipients immunized with NP-conjugated to ovalbumin (NP-OVA) developed germinal centers (GCs) containing large numbers of the antigen-specific, transferred B cells (FIG. 5A, B) and produced high levels of antigen-specific IgG1 (FIG. 5C). In addition, transfection by electroporation did not affect the ability of transferred cells to enter GCs (FIG. 5D, E).

Despite having two alleles for each of the antibody chains, B cells express only one heavy and one light chain gene, a phenomenon referred to as allelic exclusion (Cebra et al., 1966; Nussenzweig et al., 1987; Pernis et al., 1965). In the absence of the present disclosure, introducing additional antibody genes would risk random combinations of heavy and light chains some of which could be self-reactive or incompatible. Thus, deletion of the endogenous chains would be desirable to prevent expression of chimeric B cell receptors (BCRs) composed of the transgene and the endogenous antibody genes. To do so, we combined endogenous Ig disruption with insertion of a transcription unit that directs expression of the heavy and light chain into the endogenous heavy chain locus.

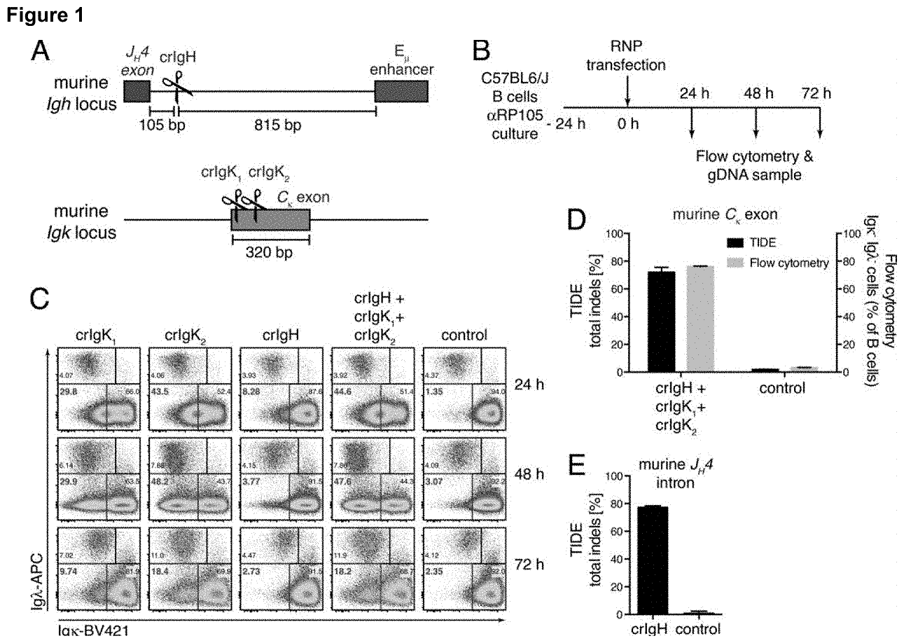

CRISPR-RNAs (crRNAs) were designed to ablate the κ-light chain because 95% of all mouse B cells express Igk (FIG. 1A). The efficiency of κ light chain deletion was measured by flow cytometry using the ratio of κ/λ cells to normalize for cell death due to BCR loss. The selected crRNAs consistently ablated Igκ expression by 70-80% of B cells as measured by flow cytometry or TIDE (Tracking the Indels by Decomposition (Brinkman et al., 2014)) analysis (FIG. 1B-D).

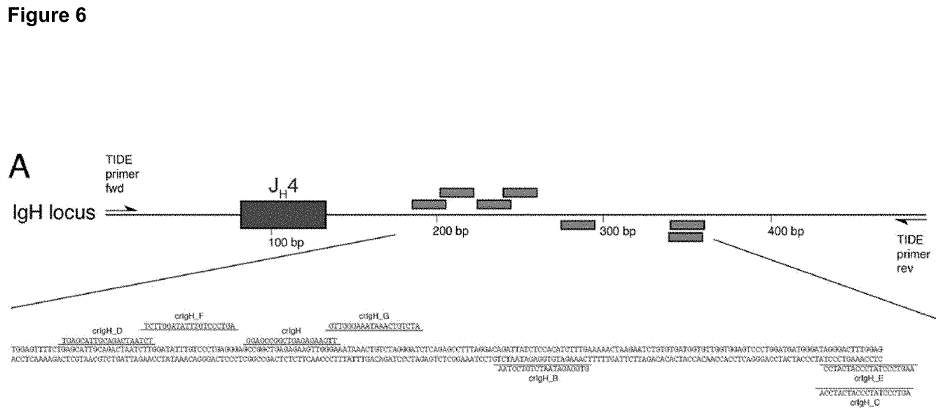

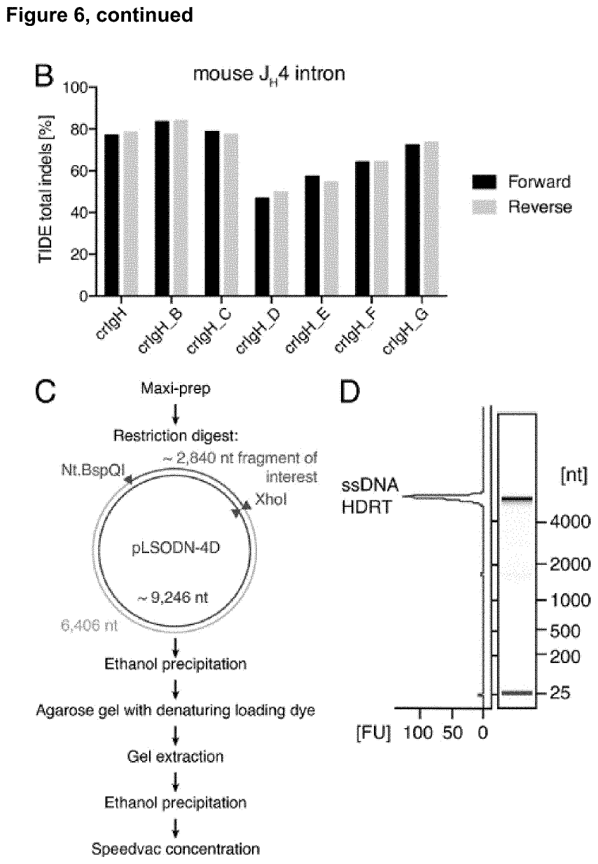

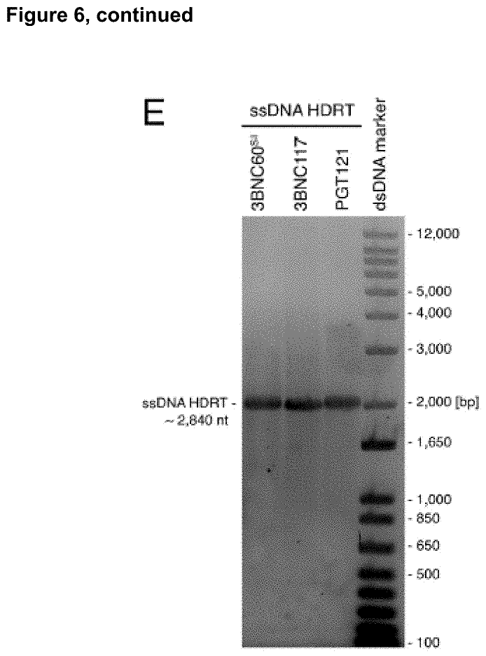

To insert a transgene into the heavy chain locus we designed crRNAs specific for the first Igh intron immediately 3′ of the endogenous VDJ_{H} gene segment, and 5′ of the Eμ enhancer. The Eu enhancer sequence is known in the art, and is located in the IGHJ6 intron, the sequence of which is described above. This position was selected to favor transgene expression and allow simultaneous disruption the endogenous heavy chain (see below and (Jacobsen et al., 2018, the disclosure of which is incorporated herein by reference)). We tested 7 crRNAs and selected a high-efficiency crRNA located 110 bp downstream of the J_{H}4 intron producing 77% indels by the TIDE assay (FIG. 1E, FIG. 6A, B). This location also allowed for sufficient homology to introduce a transgene, irrespective of the upstream VDJ rearrangement. In FIG. 6A, in the double stranded DNA sequence, the top (sense strand) of the mouse JgHJ4 intron (with crRNA targeting regions, 5′ to 3′) is:

TGGAGTTTTCTGAGCATTGCAGACTAATCTTGGATATTTGTCCCTGAGGGAGCCGGC TGAGAGAAGTTGGGAAATAAACTGTCTAGGGATCTCAGAGCCTTTAGGACAGATTA TCTCCACATCTTTGAAAAACTAAGAATCTGTGTGATGGTGTTGGTGGAGTCCCTGGA TGATGGGATAGGGACTTTGGAG (SEQ ID NO:41). In FIG. 6A, the reverse strand sequence is (with crRNA targeting regions, 5′ to 3′) is: AAGTCCCTATCCCATCATCCAGGGACTCCACCAACACCATCACACAGATTCTTAGTT TTTCAAAGATGTGGAGATAATCTGTCCTAAAGGCTCTGAGATCCCTAGACAGTTTAT TTCCCAACTTCTCTCAGCCGGCTCCCTCAGGGACAAATATCCAAGATTAGTCTGCAA TGCTCAGAAAACTCCA (SEQ ID NO:42). The SEQ ID for each of the crRNA sequences (shown as DNA) in FIG. 6A above and below the double stranded sequence are as described for the same crRNA sequences and names as in Table 1.

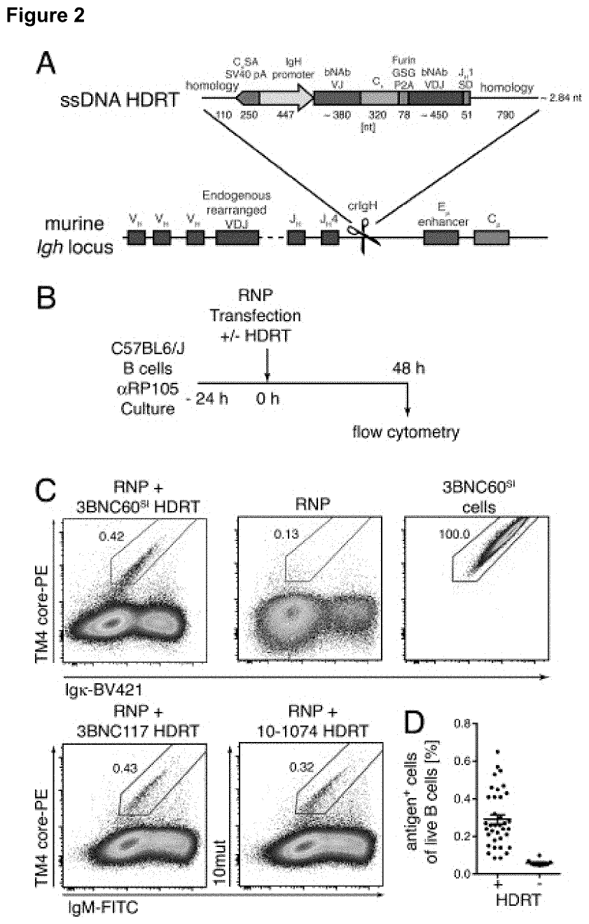

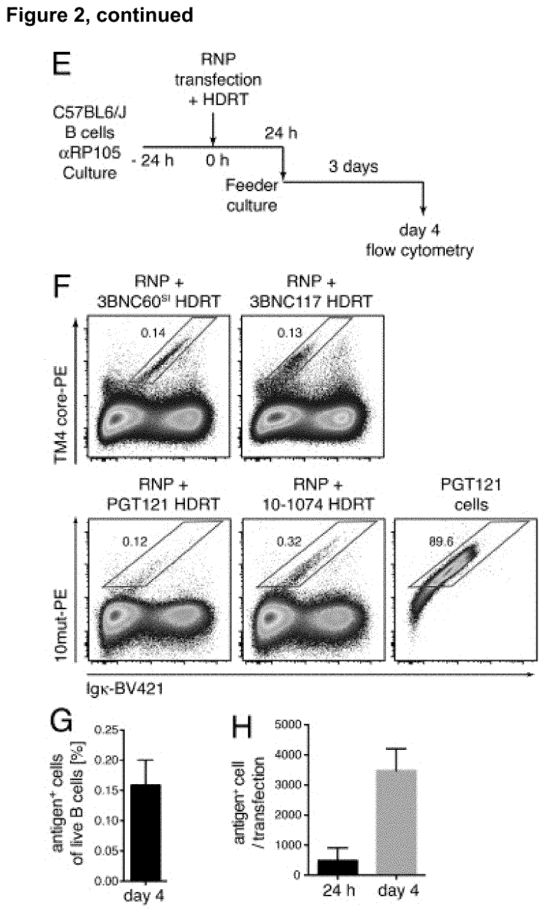

The homology-directed repair template is described above. In an embodiment, it is composed of a splice acceptor (SA) stop cassette to terminate transcription of upstream rearranged VDJ_{H}, and a V_{H}-gene promoter followed by cDNAs encoding Igk, a P2A self-cleaving sequence, and IgV_{H} with a J_{H}1 splice donor (SD) site (FIG. 2A). This design disrupts expression of the endogenous locus, while encoding a transcription unit directing expression of the introduced heavy and light chain under control of endogenous Igh gene regulatory elements. In addition, it preserves splicing of the transgenic IgV_{H} into the endogenous constant regions allowing for expression of membrane and secreted forms of the antibody as wells as different isotypes by class switch recombination. Finally, correctly targeted cells are readily identified and enumerated by flow cytometry because they bind to cognate antigen.

A number of methods for producing ssDNA homology directed repair templates (HDRTs) were compared. The most reproducible and least cytotoxic involved digestion of plasmids with sequence-specific nickases, and ssDNA purification by agarose gel electrophoresis (FIG. 6C-E) (Roth et al., 2018; Yoshimi et al., 2016).

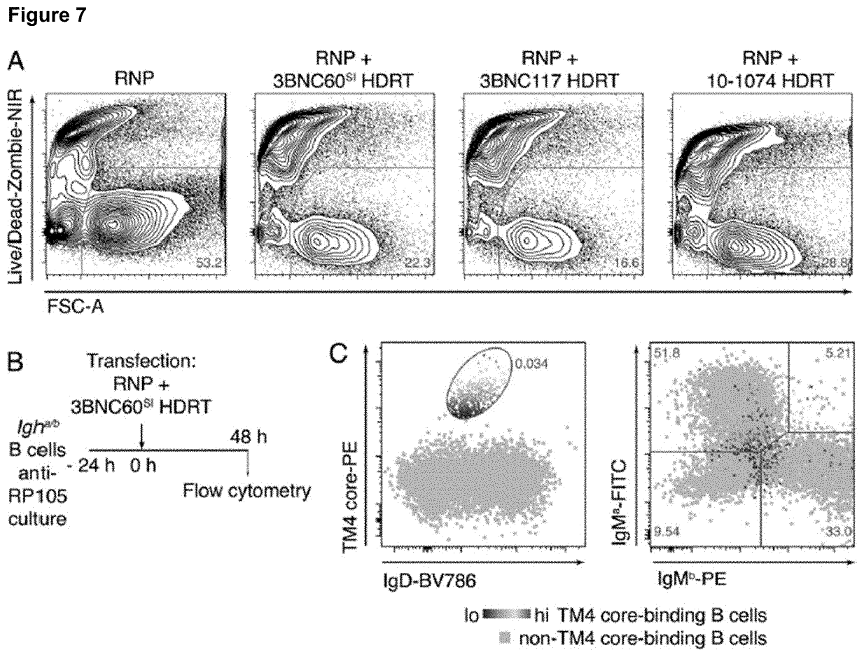

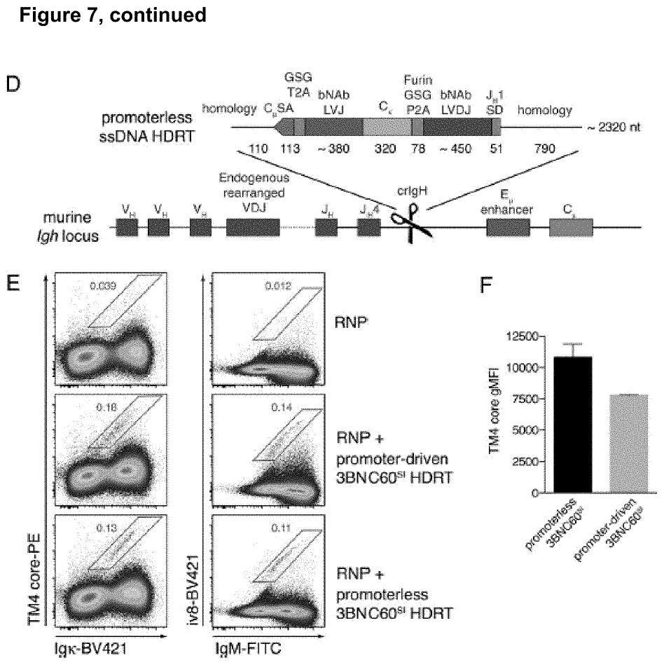

Co-transfection of the ssDNA template with pre-assembled Cas9 ribonucleoproteins (RNPs) containing the crRNAs resulted in expression of the encoded anti-HIV antibody in 0.1-0.4% of mouse B cells by antigen-specific flow cytometry using antigens TM4 core (McGuire et al., 2014; McGuire et al., 2016) or 10mut (Steichen et al., 2016) (FIG. 2C,D, FIG. 7 A). Transgene expression was stable over the entire culture period of 3 days on feeder cells (Kuraoka et al., 2016), during which the overall number of B cells expanded by 6 to 20-fold (FIG. 2E-H). However, expression of transgenic antibodies differed depending on the antibody and were generally reflective of their expression in knock-in mouse models (FIG. 2C, F) (Dosenovic et al., 2018; Dosenovic et al., 2015; Escolano et al., 2016; McGuire et al., 2016; Steichen et al., 2016).

To determine whether edited cells are allelically excluded at the heavy chain locus we transfected Igh^{a/b} B cells with 3BNC60^{SI}, a chimeric antibody composed of the mature heavy chain and germline light chain of the anti-HIV bNAb 3BNC60 (FIG. 7B, C). The majority of edited cells expressing the 3BNC60^{SI} transgene, expressed it using either Igm^{a} or Igm^{b} allele as determined by flow cytometry. Only 5.21% of 3BNC60^{SI}-expressing B cells showed co-expression of both IgM^{a} and IgM^{b} indicative of allelic inclusion of the endogenous allele or successful integration of the transgene into both alleles. Thus, the majority of edited B cells express only the transgene.

Promoter containing expression cassettes have the potential to cause unwanted ectopic gene expression or allelic inclusion since they can be expressed from either the rearranged or germline IgH locus. To address these potential problems we designed a smaller, promoterless antibody expression cassette that depends on integration into a rearranged IgH allele for expression (FIG. 7D). Cell surface expression of the 3BNC60^{SI} from the promoterless construct was higher than the promoter-driven version (FIG. 7E, F). Thus, the smaller promoterless, and potentially safer construct efficiently directs knock-in antibody expression.

Without intending to be bound by any particular theory, we conclude that mature mouse B cells can be edited in vitro to produce anti-HIV-1 bNAbs from the Igh locus.

### EXAMPLE 2

Antibody Gene Editing in Human B Cells

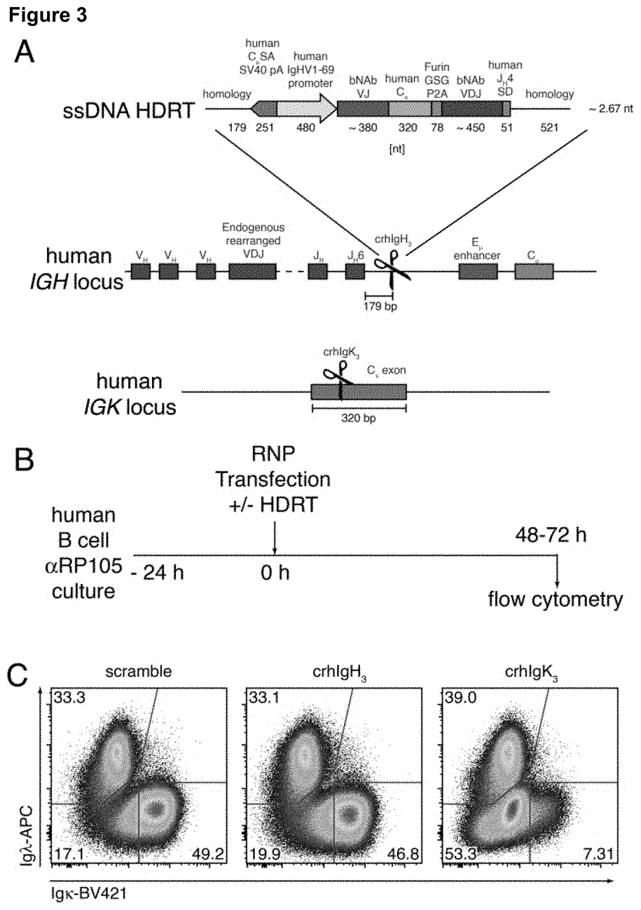

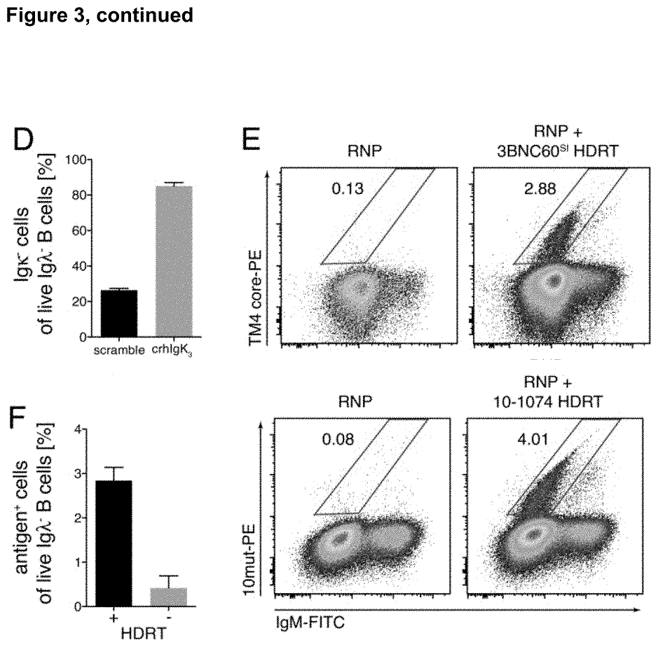

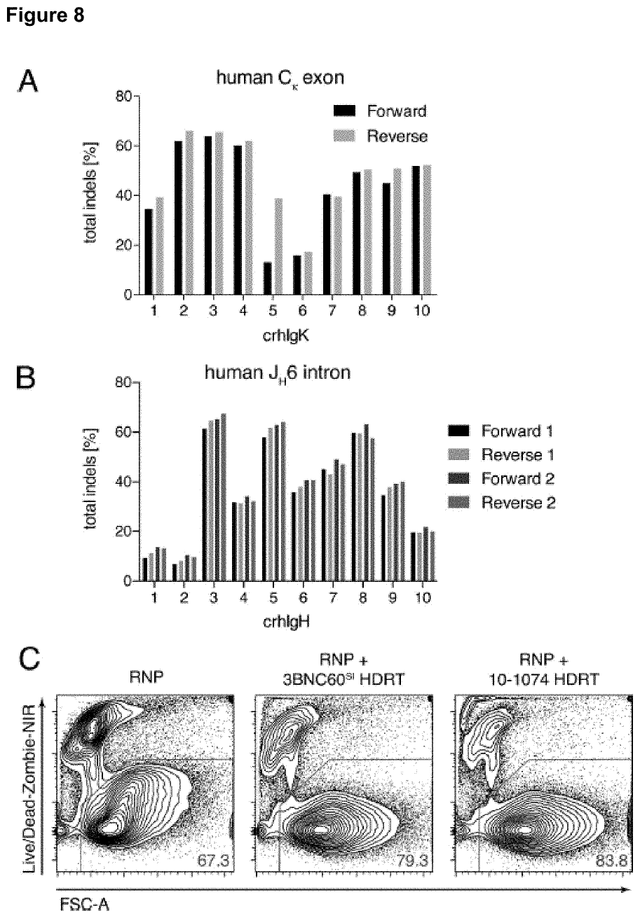

To determine whether this method could be adapted to edit human B cells we isolated them from peripheral blood of healthy volunteers and activated them using an anti-human RP105 antibody (Miura et al., 1998). Analogous crRNAs were selected for targeting the human IGKC and the first intron 3′ of IGHJ6 (FIG. 3A-D, FIG. 8A, B). The best IGKC-targeting crRNA caused 85% of κ-bearing B cells to lose BCR expression, whereas λ-bearing cells increased proportionally indicating that they were unaffected. TIDE analysis of the J_{H}6 intron sequences showed that the most efficient crRNA induced 64% indels. In conclusion, activation of human, primary B cells with anti-RP105 allows efficient generation of indels using Cas9 RNPs.

To target bNAbs into the human J_{H}6 intron we adapted the ssDNA HDRT and replaced mouse with human homology arms, the human Cμ splice acceptor, the human IGHV1-69 promoter, a codon-modified human IGKC constant region to avoid targeting by crRNAs and the human J_{H}4 splice donor (FIG. 3A). In contrast to mouse cells, 2.9-4% of λ− B cells expressed 3BNC60^{SI} or 10-1074 antibodies respectively as determined by flow cytometry using the cognate antigen (FIG. 3E, F). Thus, the efficiency of transgene integration is at least 10-times higher in human B cells. Furthermore, viability was also higher in human B cells, ranging from 60 to 85% of live cells after transfection (FIG. 8C).

Without intending to be bound by any particular theory, we conclude that primary human B cells can be edited by CRISPR/Cas9 to express anti-HIV bNAbs, and that this is significantly more efficient than in mouse B cells.

### EXAMPLE 3

Adoptive Transfer of Antibody-Edited B Cells

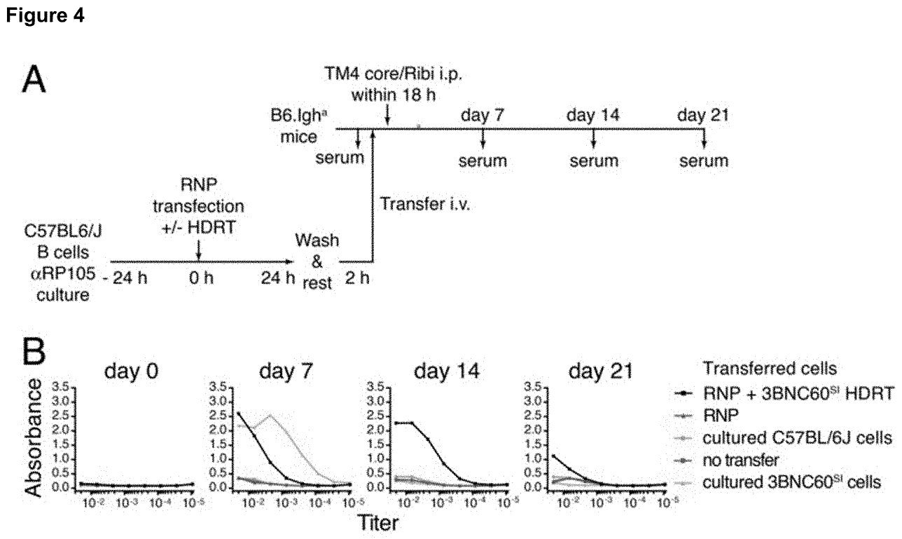

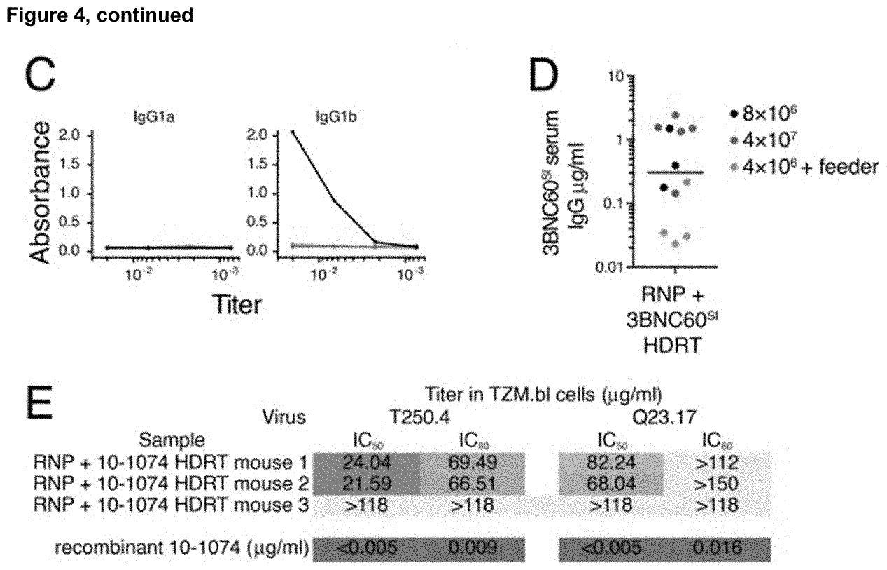

To determine whether edited B cells can participate in immune responses, we adoptively transferred mouse 3BNC60^{SI}-edited Igh^{b} B cells, into congenically-marked Igh^{a} wild type mice and immunized with the high-affinity, cognate antigen TM4 core in Ribi adjuvant (FIG. 4A). Transgene-specific responses were detected using anti-idiotypic antibodies as an initial capture reagent in ELISA. Similar to endogenous humoral immune responses, transgenic antibodies were detected on day 7 after immunization, they peaked at day 14 and started to decrease by day 21 (FIG. 4B, C). Importantly, the transgenic immune response included secondary isotypes indicating that the re-engineered locus supports class switch recombination (FIG. 4C). Finally, the magnitude of the response was directly correlated to the number of transferred cells. However, prolonged in vitro culture under the conditions tested decreased the efficiency of antibody production in vivo (FIG. 4D).

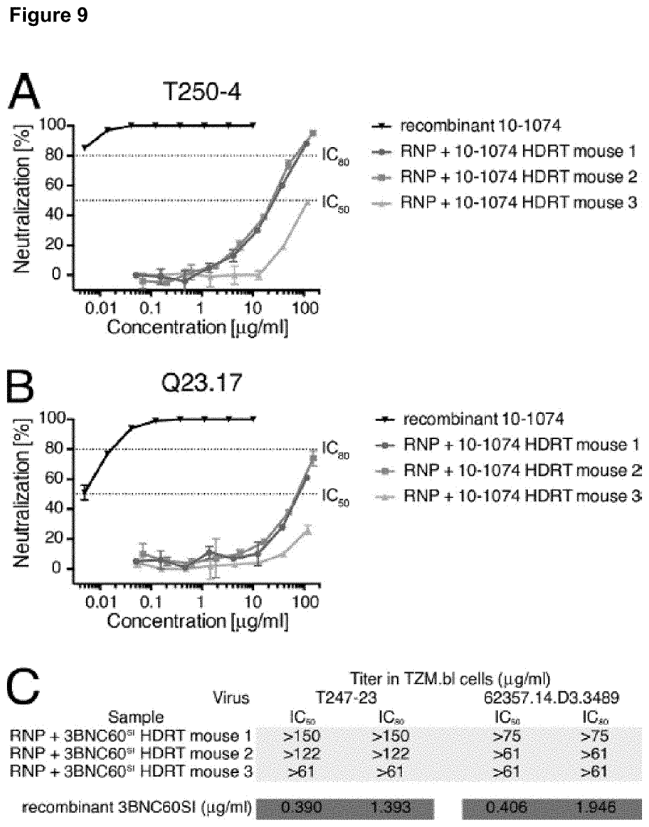

To determine whether the transferred cells retained the ability to produce neutralizing antibodies we used B cells that were edited to produce 10-1074, a potent bNAb, or 3BNC60^{SI} a chimeric antibody with limited neutralizing activity (Dosenovic et al., 2018; Mouquet et al., 2012). 4×10⁷ transfected B cells were transferred into wild type Igh^{a} mice that were subsequently immunized with the appropriate cognate antigen 10mut (Steichen et al., 2016) or TM4 core (Dosenovic et al., 2018; Dosenovic et al., 2015; McGuire et al., 2014; McGuire et al., 2016). IgG was purified from the serum of 3 mice that received an estimated ˜103 edited B cells expressing 10-1074 or 3BNC60^{SI}. The purified serum antibodies were tested for neutralizing activity in the TZM-b1 assay (Montefiori, 2005). Two of the 3 mice that received 10-1074 edited cells showed IC50s of 21.59 μg/mL and a third reached 49% neutralization at 118 μg/mL (corresponding to approximately 1:500 and 1:100 dilution of serum, FIG. 4E, FIG. 9A, B). As expected, neutralizing activity was not detected in mice receiving 3BNC60^{SI} because this antibody is 2-3 orders of magnitude less potent against the tested viral strains than 10-1074 (FIG. 9C).

Without intending to be bound by theory, we conclude that edited B cells can be recruited into immune responses and produce sufficient antibody to confer potentially protective levels of humoral immunity (Shingai et al., 2014).

### EXAMPLE 4

This Example provides a description of the materials and methods used to produce the foregoing results.

crRNA Design

crRNAs were designed with the MIT guide design tool (crispr.mit.edu), CHOPCHOP (chopchop.cbu.uib.no) and the IDT crRNA design tool (www.idtdna.com). Designs were synthesized by IDT as Alt-R CRISPR-Cas9 crRNAs. crRNA sequences are listed in Table 1 as DNA sequences, these sequences include sequences with substitution of U for T.

TABLE 1
crRNA sequences

| | crRNA sequence | | SEQ |
|---|---|---|---|
| Name | without PAM | Locus | ID NO |
| crIgK₁ | GTTCAAGAAGCACACGACTG | mouse Igkc | 1 |
| crIgK₂ | GTTAACTGCTCACTGGATGG | mouse Igkc | 2 |
| crIgH | GGAGCCGGCTGAGAGAAGTT | mouse J_{H}4 intron | 3 |
| crIgH_B | GTGGAGATAATCTGTCCTAA | mouse J_{H}4 intron | 4 |
| crIgH_C | AGTCCCTATCCCATCATCCA | mouse J_{H}4 intron | 5 |
| crIgH_D | TGAGCATTGCAGACTAATCT | mouse J_{H}4 intron | 6 |
| crIgH_E | AAGTCCCTATCCCATCATCC | mouse J_{H}4 intron | 7 |
| crIgH_F | TCTTGGATATTTGTCCCTGA | mouse J_{H}4 intron | 8 |
| crIgH_G | GTTGGGAAATAAACTGTCTA | mouse J_{H}4 intron | 9 |
| crhIgK₁ | GGTGGATAACGCCCTCCAAT | human IGKC | 10 |
| crhIgK₂ | GTGGATAACGCCCTCCAATC | human IGKC | 11 |
| crhIgK₃ | CTGGGAGTTACCCGATTGGA | human IGKC | 12 |
| crhIgK₄ | CCTCCAATCGGGTAACTCCC | human IGKC | 13 |
| crhIgK₅ | ATCCACCTTCCACTGTACTT | human IGKC | 14 |
| crhIgK₆ | TTCAACTGCTCATCAGATGG | human IGKC | 15 |
| crhIgK₇ | GATTTCAACTGCTCATCAGA | human IGKC | 16 |
| crhIgK₈ | TGGGATAGAAGTTATTCAGC | human IGKC | 17 |
| crhIgK₉ | ATTCAGCAGGCACACAACAG | human IGKC | 18 |
| crhIgK₁₀ | GGCCAAAGTACAGTGGAAGG | human IGKC | 19 |
| crhIgH₁ | GTCCTCGGGGCATGTTCCGA | human J_{H}6 intron | 20 |
| crhIgH₂ | TCCTCGGGGCATGTTCCGAG | human J_{H}6 intron | 21 |
| crhIgH₃ | AGGCATCGGAAAATCCACAG | human J_{H}6 intron | 22 |
| crhIgH₄ | CTCAGGTTGGGTGCGTCTGA | human J_{H}6 intron | 23 |
| crhIgH₅ | ACGAGATGCCTGAACAAACC | human J_{H}6 intron | 24 |
| crhIgH₆ | ACCTGAGTCCCATTTTCCAA | human J_{H}6 intron | 25 |
| crhIgH₇ | TCAGCCATCACTAAGACCCC | human J_{H}6 intron | 26 |
| crhIgH₈ | CAAACCAGGGGTCTTAGTGA | human J_{H}6 intron | 27 |
| crhIgH₉ | CTAAGACCCCTGGTTTGTTC | human J_{H}6 intron | 28 |
| crhIgH₁₀ | TCAGGCATCTCGTCCAAATG | human J_{H}6 intron | 29 |

ssDNA HDRT Preparation

HDRT sequences, listed in Table 2, were synthesized as gBlocks (IDT) and cloned using NheI and XhoI (NEB) into vector pLSODN-4D from the long ssDNA preparation kit (BioDynamics Laboratories, Cat. #DS620). ssDNA was prepared following the manufacturer's instructions with the following modifications: In brief, 2.4 mg sequence verified vector was digested at 2 μg/μL in NEB 3.1 buffer with 1200 U Nt.BspQI for 1 h at 50° C. followed by addition of 2400 U XhoI (NEB) and incubation for 1 hat 37° C. Digests were desalted by ethanol precipitation and resuspended in water at <1 μg/μL. An equal volume of formamide gel-loading buffer (95% de-ionized formamide, 0.025% bromophenol blue, 0.025% xylene cyanol, 0.025 SDS, 18 mM EDTA) was added and heated to 70° C. for 5 min to denature dsDNA. Denatured samples were immediately loaded into dye-free 1% agarose gels in TAE and run at 100 V for 3 h. Correctly sized bands were identified by partial post-stain with GelRed (Biotium), then excised and column purified (Machery Nagel Cat. #740610.20 or 740609.250) according to the manufacturer's instructions. Eluate was ethanol precipitated, resuspended in water, adjusted to 2.5 μg/μL and stored at −20° C.

TABLE 2
gBlock sequences of HDRTs (Table 2 is reproduced as
FIG. 10, with the color nucleotide key included).

3BNC60^{SI}, mouse (SEQ ID NO: 30)
GCATAGCTAGCGCTCTTCAGTAAGAATGGCCTCTCCAGGTCTTTATTTTTAACCTTTGTTA
TGGAGTTTTCTGAGCATTGCAGACTAATCTTGGATATTTGTCCCTGAGGGAGCCGGCTGAG
AGAAGTTAAGAGTAGCAACAAGGAAATAGCAGGGTGTAGAGGGATCTCCTGTCTGACAG
GAGGCAAGAAGACAGATTCTTACCCCTCCATTTCTCTTTTATCCCTCTCTGGTCCTCAGAG
AGTTAGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTA
TCTTATCATGTCTGGTCGACAGTATGCAGAGGGCTGTATCCACTGGAGAGGATGAAGTCA
CTGAGTTGGAAAACAGAACAGGACAGGCACCTAACAAGTGGTTGCTATAGCCCACTGTTA
CCCTTTTACATGTATAGGCTCAGGATAAGCAGTGATACTGTGAGGTTTATGTGTGAGAACA
TCACAGTATAAACACATCTCAATAGAGGTCTTAGAGATCAGCACAATTAGTGAGAAGTCA
TAAACAGTAGATACTATAAGGCATAGGCTCAGCTACCTAGGGTCAGGTATCTGTGTAAAT
CTGATTGTGTATCAGGTTTAGATCAATATGACTTAGGGAGGCGAGTCATATGCAAATCTAA
GAAGACTTTAGAGAAGAAATCTGAGGCTCACCTCACATAACAGCAAGAGAGTGTCCGGTT
AGTCTCAAGGAAGACTGAGACACAGTCTTAGATATCACCATGGGATGGTCATGTATCATC
CTTTTTCTAGTAGCAACTGCAACCGGTGTACATTCTGACATCCAGATGACCCAGTCTCCAT
CCTCCCTGTCTGCATCTGTAGGAGACAGAGTCACCATCACTTGCCAGGCGAGTCAGGACAT
TAGCAACTATTTAAATTGGTATCAGCAGAAACCAGGGAAAGCCCCTAAGCTCCTGATCTA
CGATGCATCCAATTTGGAAACAGGGGTCCCATCAAGGTTCAGTGGAAGTGGATCTGGGAC
AGATTTTACTTTCACCATCAGCAGCCTGCAGCCTGAAGATATTGCAACATATTACTGTCAA
CAGTATGAGTTTATCGGCCCTGGGACCAAAGTGGATATCAAACGGGCTGATGCTGCACCA
ACTGTATCCATCTTCTCACCATCCAGTGAGCAGTTAACATCTGGAGGTGCTTCAGTCGTGT
GCTTCTTGAACAACTTCTACCCCAAAGACATCAATGTCAAGTGGAAGATTGATGGCAGTG
AACGACAAAATGGCGTCCTGAACAGTTGGACTGATCAGGACAGCAAAGACAGCACCTAC
AGCATGAGCAGCACCCTCACGTTGACCAAGGACGAGTATGAACGACATAACAGCTATACC
TGTGAGGCCACTCACAAGACATCAACTTCACCCATTGTCAAGAGCTTCAACAGGAATGAG
TGTAGGCGGAAGCGGGGGTCAGGAGCAACCAACTTTTCTCTGCTGAAGCAAGCCGGGGAC
GTAGAGGAAAACCCCGGACCCATGGGATGGTCATGTATCATCCTTTTTCTAGTAGCAACTG
CAACCGGTGTACATTCTCAGGTCCATTTGTCACAGTCTGGGGCAGCGGTGACGAAGCCCG
GGGCCTCAGTGAGAGTCTCCTGCGAGGCTTCCGGATACAAGATTAGTGACCACTTTATTCA
TTGGTGGCGACAGGCCCCAGGACAGGGCCTTCAGTGGGTGGGGTGGATCAATCCTAAGAC
TGGTCAGCCAAACAATCCTCGTCAATTTCAGGGTAGAGTCAGTCTGACTCGACAGGCGTC
GTGGGACTTTGACACATATTCCTTTTACATGGACCTCAAGGCAGTAAGATCGGACGACAC
GGCCATTTATTTCTGTGCGCGACAACGCAGCGACTTTTGGGATTTCGACGTCTGGGGCAGC
GGCACGCAGGTCACTGTCTCGTCAGGTAAGCTGGCTTTTTTCTTTCTGCACATTCCATTCTG
AAACGGGATCGATTGGGAAATAAACTGTCTAGGGATCTCAGAGCCTTTAGGACAGATTAT
CTCCACATCTTTGAAAAACTAAGAATCTGTGTGATGGTGTTGGTGGAGTCCCTGGATGATG
GGATAGGGACTTTGGAGGCTCATTTGAAGAAGATGCTAAAACAATCCTATGGCTGGAGGG
ATAGTTGGGGCTGTAGTTGGAGATTTTCAGTTTTTAGAATAAAAGTATTAGTTGTGGAATA
TACTTCAGGACCACCTCTGTGACAGCATTTATACAGTATCCGATGCATAGGGACAAAGAG
TGGAGTGGGGCACTTTCTTTAGATTTGTGAGGAATGTTCCGCACTAGATTGTTTAAAACTT
CATTTGTTGGAAGGAGAGCTGTCTTAGTGATTGAGTCAAGGGAGAAAGGCATCTAGCCTC
GGTCTCAAAAGGGTAGTTGCTGTCTAGAGAGGTCTGGTGGAGCCTGCAAAAGTCCAGCTT
TCAAAGGAACACAGAAGTATGTGTATGGAATATTAGAAGATGTTGCTTTTACTCTTAAGTT
GGTTCCTAGGAAAAATAGTTAAATACTGTGACTTTAAAATGTGAGAGGGTTTTCAAGTACT
CATTTTTTTAAATGTCCAAAATTCTTGTCAATCAGTTTGAGGTCTTGTTTGTGTAGAACTGA
TATTACTTAAAGTTTAACCGAGGAATGGGAGTGAGGCTCTCTCATAACCTATTCAGAACTG
ACTTTTAACAATAATAAATTAAGTTTCAAATATTTTTAAATGAATTGAGCAATGTTGAGTT
GGAGTCAAGATGGCCTCGAGGAAT
Promoterless 3BNC60^{SI}, mouse (SEQ ID NO: 31)
GCATAGCTAGCGCTCTTCAGTAAGAATGGCCTCTCCAGGTCTTTATTTTTAACCTTTGTTA
TGGAGTTTTCTGAGCATTGCAGACTAATCTTGGATATTTGTCCCTGAGGGAGCCGGCTGAG
AGAAGTTAAGAGTAGCAACAAGGAAATAGCAGGGTGTAGAGGGATCTCCTGTCTGACAG
GAGGCAAGAAGACAGATTCTTACCCCTCCATTTCTCTTTTATCCCTCTCTGGTCCTCAGAG
GGAAGCGGAGAGGGCAGAGGAAGTCTGCTAACATGCGGTGACGTCGAGGAGAATCCTGG
ACCTATGGGATGGTCATGTATCATCCTTTTTCTAGTAGCAACTGCAACCGGTGTACATTCT
GACATCCAGATGACCCAGTCTCCATCCTCCCTGTCTGCATCTGTAGGAGACAGAGTCACCA
TCACTTGCCAGGCGAGTCAGGACATTAGCAACTATTTAAATTGGTATCAGCAGAAACCAG
GGAAAGCCCCTAAGCTCCTGATCTACGATGCATCCAATTTGGAAACAGGGGTCCCATCAA
GGTTCAGTGGAAGTGGATCTGGGACAGATTTTACTTTCACCATCAGCAGCCTGCAGCCTGA
AGATATTGCAACATATTACTGTCAACAGTATGAGTTTATCGGCCCTGGGACCAAAGTGGAT
ATCAAACGGGCTGATGCTGCACCAACTGTATCCATCTTCTCACCATCCAGTGAGCAGTTAA
CATCTGGAGGTGCTTCAGTCGTGTGCTTCTTGAACAACTTCTACCCCAAAGACATCAATGT
CAAGTGGAAGATTGATGGCAGTGAACGACAAAATGGCGTCCTGAACAGTTGGACTGATCA
GGACAGCAAAGACAGCACCTACAGCATGAGCAGCACCCTCACGTTGACCAAGGACGAGT
ATGAACGACATAACAGCTATACCTGTGAGGCCACTCACAAGACATCAACTTCACCCATTG
TCAAGAGCTTCAACAGGAATGAGTGTAGGCGGAAGCGGGGGTCAGGAGCAACCAACTTTT
CTCTGCTGAAGCAAGCCGGGGACGTAGAGGAAAACCCCGGACCCATGGGATGGTCATGTA
TCATCCTTTTTCTAGTAGCAACTGCAACCGGTGTACATTCTCAGGTCCATTTGTCACAGTCT
GGGGCAGCGGTGACGAAGCCCGGGGCCTCAGTGAGAGTCTCCTGCGAGGCTTCCGGATAC
AAGATTAGTGACCACTTTATTCATTGGTGGCGACAGGCCCCAGGACAGGGCCTTCAGTGG
GTGGGGTGGATCAATCCTAAGACTGGTCAGCCAAACAATCCTCGTCAATTTCAGGGTAGA
GTCAGTCTGACTCGACAGGCGTCGTGGGACTTTGACACATATTCCTTTTACATGGACCTCA
AGGCAGTAAGATCGGACGACACGGCCATTTATTTCTGTGCGCGACAACGCAGCGACTTTT
GGGATTTCGACGTCTGGGGCAGCGGCACGCAGGTCACTGTCTCGTCAGGTAAGCTGGCTTT
TTTCTTTCTGCACATTCCATTCTGAAACGGGATCGATTGGGAAATAAACTGTCTAGGGATC
TCAGAGCCTTTAGGACAGATTATCTCCACATCTTTGAAAAACTAAGAATCTGTGTGATGGT
GTTGGTGGAGTCCCTGGATGATGGGATAGGGACTTTGGAGGCTCATTTGAAGAAGATGCT
AAAACAATCCTATGGCTGGAGGGATAGTTGGGGCTGTAGTTGGAGATTTTCAGTTTTTAGA
ATAAAAGTATTAGTTGTGGAATATACTTCAGGACCACCTCTGTGACAGCATTTATACAGTA
TCCGATGCATAGGGACAAAGAGTGGAGTGGGGCACTTTCTTTAGATTTGTGAGGAATGTT
CCGCACTAGATTGTTTAAAACTTCATTTGTTGGAAGGAGAGCTGTCTTAGTGATTGAGTCA
AGGGAGAAAGGCATCTAGCCTCGGTCTCAAAAGGGTAGTTGCTGTCTAGAGAGGTCTGGT
GGAGCCTGCAAAAGTCCAGCTTTCAAAGGAACACAGAAGTATGTGTATGGAATATTAGAA
GATGTTGCTTTTACTCTTAAGTTGGTTCCTAGGAAAAATAGTTAAATACTGTGACTTTAAA
ATGTGAGAGGGTTTTCAAGTACTCATTTTTTTAAATGTCCAAAATTCTTGTCAATCAGTTTG
AGGTCTTGTTTGTGTAGAACTGATATTACTTAAAGTTTAACCGAGGAATGGGAGTGAGGCT
CTCTCATAACCTATTCAGAACTGACTTTTAACAATAATAAATTAAGTTTCAAATATTTTTAA
ATGAATTGAGCAATGTTGAGTTGGAGTCAAGATGGCCTCGAGATGA
10-1074, mouse (SEQ ID NO: 32)
GCATAGCTAGCGCTCTTCAGTAAGAATGGCCTCTCCAGGTCTTTATTTTTAACCTTTGTTA
TGGAGTTTTCTGAGCATTGCAGACTAATCTTGGATATTTGTCCCTGAGGGAGCCGGCTGAG
AGAAGTTAAGAGTAGCAACAAGGAAATAGCAGGGTGTAGAGGGATCTCCTGTCTGACAG
GAGGCAAGAAGACAGATTCTTACCCCTCCATTTCTCTTTTATCCCTCTCTGGTCCTCAGAG
AGTTAGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTA
TCTTATCATGTCTGGTCGACAGTATGCAGAGGGCTGTATCCACTGGAGAGGATGAAGTCA
CTGAGTTGGAAAACAGAACAGGACAGGCACCTAACAAGTGGTTGCTATAGCCCACTGTTA
CCCTTTTACATGTATAGGCTCAGGATAAGCAGTGATACTGTGAGGTTTATGTGTGAGAACA
TCACAGTATAAACACATCTCAATAGAGGTCTTAGAGATCAGCACAATTAGTGAGAAGTCA
TAAACAGTAGATACTATAAGGCATAGGCTCAGCTACCTAGGGTCAGGTATCTGTGTAAAT
CTGATTGTGTATCAGGTTTAGATCAATATGACTTAGGGAGGCGAGTCATATGCAAATCTAA
GAAGACTTTAGAGAAGAAATCTGAGGCTCACCTCACATAACAGCAAGAGAGTGTCCGGTT
AGTCTCAAGGAAGACTGAGACACAGTCTTAGATATCACCATGGGATGGTCATGTATCATC
CTTTTTCTAGTAGCAACTGCAACCGGTGTACATTCTTCCTATGTGCGCCCGCTGTCAGTGGC
CCTGGGGGAGACGGCCAGGATTTCCTGTGGACGACAGGCCCTTGGAAGTAGAGCTGTTCA
GTGGTATCAACATAGGCCAGGCCAGGCCCCTATATTGCTCATTTATAATAATCAAGACCGG
CCCTCAGGGATCCCTGAGCGATTCTCTGGCACCCCTGATATTAATTTTGGGACCAGGGCCA
CCCTGACCATCAGCGGGGTCGAAGCCGGGGATGAAGCCGACTATTACTGTCACATGTGGG
ATAGTAGAAGTGGCTTCAGTTGGTCTTTCGGCGGGGCGACCAGGCTGACCGTCCTACGGG
CTGATGCTGCACCAACTGTATCCATCTTCTCACCATCCAGTGAGCAGTTAACATCTGGAGG
TGCTTCAGTCGTGTGCTTCTTGAACAACTTCTACCCCAAAGACATCAATGTCAAGTGGAAG
ATTGATGGCAGTGAACGACAAAATGGCGTCCTGAACAGTTGGACTGATCAGGACAGCAAA
GACAGCACCTACAGCATGAGCAGCACCCTCACGTTGACCAAGGACGAGTATGAACGACAT
AACAGCTATACCTGTGAGGCCACTCACAAGACATCAACTTCACCCATTGTCAAGAGCTTCA
ACAGGAATGAGTGTAGGCGGAAGCGGGGGTCAGGAGCAACCAACTTTTCTCTGCTGAAGC
AAGCCGGGGACGTAGAGGAAAACCCCGGACCCATGGGATGGTCATGTATCATCCTTTTTC
TAGTAGCAACTGCAACCGGTGTACATTCTCAGGTGCAGCTGCAGGAGTCGGGCCCAGGAC
TGGTGAAACCTTCGGAGACCCTGTCCGTCACCTGCAGTGTCTCTGGAGATTCCATGAATAA
TTACTACTGGACTTGGATCCGGCAGTCCCCCGGAAAGGGACTGGAGTGGATAGGCTATAT
CTCTGACAGAGAATCAGCGACTTACAACCCCTCCCTCAATAGTCGAGTCGTCATATCACGA
GACACGTCGAAAAACCAATTGTCCCTAAAATTAAACTCCGTCACCCCTGCGGACACGGCC
GTCTATTACTGTGCGACAGCGCGCCGAGGACAGAGGATTTATGGAGTGGTTTCCTTTGGAG
AGTTCTTCTACTACTACTCCATGGACGTCTGGGGCAAGGGGACCACGGTCACCGTCTCCTC
AGGTAAGCTGGCTTTTTTCTTTCTGCACATTCCATTCTGAAACGGGATCGATTGGGAAATA
AACTGTCTAGGGATCTCAGAGCCTTTAGGACAGATTATCTCCACATCTTTGAAAAACTAAG
AATCTGTGTGATGGTGTTGGTGGAGTCCCTGGATGATGGGATAGGGACTTTGGAGGCTCAT
TTGAAGAAGATGCTAAAACAATCCTATGGCTGGAGGGATAGTTGGGGCTGTAGTTGGAGA
TTTTCAGTTTTTAGAATAAAAGTATTAGTTGTGGAATATACTTCAGGACCACCTCTGTGAC
AGCATTTATACAGTATCCGATGCATAGGGACAAAGAGTGGAGTGGGGCACTTTCTTTAGA
TTTGTGAGGAATGTTCCGCACTAGATTGTTTAAAACTTCATTTGTTGGAAGGAGAGCTGTC
TTAGTGATTGAGTCAAGGGAGAAAGGCATCTAGCCTCGGTCTCAAAAGGGTAGTTGCTGT
CTAGAGAGGTCTGGTGGAGCCTGCAAAAGTCCAGCTTTCAAAGGAACACAGAAGTATGTG
TATGGAATATTAGAAGATGTTGCTTTTACTCTTAAGTTGGTTCCTAGGAAAAATAGTTAAA
TACTGTGACTTTAAAATGTGAGAGGGTTTTCAAGTACTCATTTTTTTAAATGTCCAAAATTC
TTGTCAATCAGTTTGAGGTCTTGTTTGTGTAGAACTGATATTACTTAAAGTTTAACCGAGG
AATGGGAGTGAGGCTCTCTCATAACCTATTCAGAACTGACTTTTAACAATAATAAATTAAG
TTTCAAATATTTTTAAATGAATTGAGCAATGTTGAGTTGGAGTCAAGATGGCCTCGAGGAA
T
3BNC117, mouse (SEQ ID NO: 33)
GCATAGCTAGCGCTCTTCAGTAAGAATGGCCTCTCCAGGTCTTTATTTTTAACCTTTGTTA
TGGAGTTTTCTGAGCATTGCAGACTAATCTTGGATATTTGTCCCTGAGGGAGCCGGCTGAG
AGAAGTTAAGAGTAGCAACAAGGAAATAGCAGGGTGTAGAGGGATCTCCTGTCTGACAG
GAGGCAAGAAGACAGATTCTTACCCCTCCATTTCTCTTTTATCCCTCTCTGGTCCTCAGAG
AGTTAGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTA
TCTTATCATGTCTGGTCGACAGTATGCAGAGGGCTGTATCCACTGGAGAGGATGAAGTCA
CTGAGTTGGAAAACAGAACAGGACAGGCACCTAACAAGTGGTTGCTATAGCCCACTGTTA
CCCTTTTACATGTATAGGCTCAGGATAAGCAGTGATACTGTGAGGTTTATGTGTGAGAACA
TCACAGTATAAACACATCTCAATAGAGGTCTTAGAGATCAGCACAATTAGTGAGAAGTCA
TAAACAGTAGATACTATAAGGCATAGGCTCAGCTACCTAGGGTCAGGTATCTGTGTAAAT
CTGATTGTGTATCAGGTTTAGATCAATATGACTTAGGGAGGCGAGTCATATGCAAATCTAA
GAAGACTTTAGAGAAGAAATCTGAGGCTCACCTCACATAACAGCAAGAGAGTGTCCGGTT
AGTCTCAAGGAAGACTGAGACACAGTCTTAGATATCACCATGGGATGGTCATGTATCATC
CTTTTTCTAGTAGCAACTGCAACCGGTGTACATTCTGACATCCAGATGACCCAGTCTCCAT
CCTCCCTGTCTGCCTCTGTGGGAGATACCGTCACTATCACTTGCCAGGCAAACGGCTACTT
AAATTGGTATCAACAGAGGCGAGGGAAAGCCCCAAAACTCCTGATCTACGATGGGTCCAA
ATTGGAAAGAGGGGTCCCATCAAGGTTCAGTGGAAGAAGATGGGGGCAAGAATATAATC
TGACCATCAACAATCTGCAGCCCGAAGACATTGCAACATATTTTTGTCAAGTGTATGAGTT
TGTCGTCCCTGGGACCAGACTGGATTTGAAACGGGCTGATGCTGCACCAACTGTATCCATC
TTCTCACCATCCAGTGAGCAGTTAACATCTGGAGGTGCTTCAGTCGTGTGCTTCTTGAACA
ACTTCTACCCCAAAGACATCAATGTCAAGTGGAAGATTGATGGCAGTGAACGACAAAATG
GCGTCCTGAACAGTTGGACTGATCAGGACAGCAAAGACAGCACCTACAGCATGAGCAGCA
CCCTCACGTTGACCAAGGACGAGTATGAACGACATAACAGCTATACCTGTGAGGCCACTC
ACAAGACATCAACTTCACCCATTGTCAAGAGCTTCAACAGGAATGAGTGTAGGCGGAAGC
GGGGGTCAGGAGCAACCAACTTTTCTCTGCTGAAGCAAGCCGGGGACGTAGAGGAAAACC
CCGGACCCATGGGATGGTCATGTATCATCCTTTTTCTAGTAGCAACTGCAACCGGTGTACA
TTCTCAGGTCCAATTGTTACAGTCTGGGGCAGCGGTGACGAAGCCCGGGGCCTCAGTGAG
AGTCTCCTGCGAGGCTTCTGGATACAACATTCGTGACTACTTTATTCATTGGTGGCGACAG
GCCCCAGGACAGGGCCTTCAGTGGGTGGGATGGATCAATCCTAAGACAGGTCAGCCAAAC
AATCCTCGTCAATTTCAGGGTAGAGTCAGTCTGACTCGACACGCGTCGTGGGACTTTGACA
CATTTTCCTTTTACATGGACCTGAAGGCACTAAGATCGGACGACACGGCCGTTTATTTCTG
TGCGCGACAGCGCAGCGACTATTGGGATTTCGACGTCTGGGGCAGTGGAACCCAGGTCAC
TGTCTCGTCAGGTAAGCTGGCTTTTTTCTTTCTGCACATTCCATTCTGAAACGGGATCGATT
GGGAAATAAACTGTCTAGGGATCTCAGAGCCTTTAGGACAGATTATCTCCACATCTTTGAA
AAACTAAGAATCTGTGTGATGGTGTTGGTGGAGTCCCTGGATGATGGGATAGGGACTTTG
GAGGCTCATTTGAAGAAGATGCTAAAACAATCCTATGGCTGGAGGGATAGTTGGGGCTGT
AGTTGGAGATTTTCAGTTTTTAGAATAAAAGTATTAGTTGTGGAATATACTTCAGGACCAC
CTCTGTGACAGCATTTATACAGTATCCGATGCATAGGGACAAAGAGTGGAGTGGGGCACT
TTCTTTAGATTTGTGAGGAATGTTCCGCACTAGATTGTTTAAAACTTCATTTGTTGGAAGG
AGAGCTGTCTTAGTGATTGAGTCAAGGGAGAAAGGCATCTAGCCTCGGTCTCAAAAGGGT
AGTTGCTGTCTAGAGAGGTCTGGTGGAGCCTGCAAAAGTCCAGCTTTCAAAGGAACACAG
AAGTATGTGTATGGAATATTAGAAGATGTTGCTTTTACTCTTAAGTTGGTTCCTAGGAAAA
ATAGTTAAATACTGTGACTTTAAAATGTGAGAGGGTTTTCAAGTACTCATTTTTTTAAATG
TCCAAAATTCTTGTCAATCAGTTTGAGGTCTTGTTTGTGTAGAACTGATATTACTTAAAGTT
TAACCGAGGAATGGGAGTGAGGCTCTCTCATAACCTATTCAGAACTGACTTTTAACAATA
ATAAATTAAGTTTCAAATATTTTTAAATGAATTGAGCAATGTTGAGTTGGAGTCAAGATGG
CCTCGAGGAAT
PGT121, mouse (SEQ ID NO: 34)
GCATAGCTAGCGCTCTTCAGTAAGAATGGCCTCTCCAGGTCTTTATTTTTAACCTTTGTTA
TGGAGTTTTCTGAGCATTGCAGACTAATCTTGGATATTTGTCCCTGAGGGAGCCGGCTGAG
AGAAGTTAAGAGTAGCAACAAGGAAATAGCAGGGTGTAGAGGGATCTCCTGTCTGACAG
GAGGCAAGAAGACAGATTCTTACCCCTCCATTTCTCTTTTATCCCTCTCTGGTCCTCAGAG
AGTTAGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTA
TCTTATCATGTCTGGTCGACAGTATGCAGAGGGCTGTATCCACTGGAGAGGATGAAGTCA
CTGAGTTGGAAAACAGAACAGGACAGGCACCTAACAAGTGGTTGCTATAGCCCACTGTTA
CCCTTTTACATGTATAGGCTCAGGATAAGCAGTGATACTGTGAGGTTTATGTGTGAGAACA
TCACAGTATAAACACATCTCAATAGAGGTCTTAGAGATCAGCACAATTAGTGAGAAGTCA
TAAACAGTAGATACTATAAGGCATAGGCTCAGCTACCTAGGGTCAGGTATCTGTGTAAAT
CTGATTGTGTATCAGGTTTAGATCAATATGACTTAGGGAGGCGAGTCATATGCAAATCTAA
GAAGACTTTAGAGAAGAAATCTGAGGCTCACCTCACATAACAGCAAGAGAGTGTCCGGTT
AGTCTCAAGGAAGACTGAGACACAGTCTTAGATATCACCATGGGATGGTCATGTATCATC
CTTTTTCTAGTAGCAACTGCAACCGGTGTACATTCTTCCGATATATCTGTGGCCCCAGGAG
AGACGGCCAGGATTTCCTGTGGGGAAAAGAGCCTTGGAAGTAGAGCTGTACAATGGTATC
AACACAGGGCCGGCCAGGCCCCCTCTTTAATCATATATAATAATCAGGACCGGCCCTCAG
GGATCCCTGAGCGATTCTCTGGCTCCCCTGACTCCCCTTTTGGGACCACGGCCACCCTGAC
CATCACCAGTGTCGAAGCCGGGGATGAGGCCGACTATTACTGTCATATATGGGATAGTAG
AGTTCCCACCAAATGGGTCTTCGGCGGAGGGACCACGCTGACCGTGTTACGGGCTGATGC
TGCACCAACTGTATCCATCTTCTCACCATCCAGTGAGCAGTTAACATCTGGAGGTGCTTCA
GTCGTGTGCTTCTTGAACAACTTCTACCCCAAAGACATCAATGTCAAGTGGAAGATTGATG
GCAGTGAACGACAAAATGGCGTCCTGAACAGTTGGACTGATCAGGACAGCAAAGACAGC
ACCTACAGCATGAGCAGCACCCTCACGTTGACCAAGGACGAGTATGAACGACATAACAGC
TATACCTGTGAGGCCACTCACAAGACATCAACTTCACCCATTGTCAAGAGCTTCAACAGG
AATGAGTGTAGGCGGAAGCGGGGGTCAGGAGCAACCAACTTTTCTCTGCTGAAGCAAGCC
GGGGACGTAGAGGAAAACCCCGGACCCATGGGATGGTCATGTATCATCCTTTTTCTAGTA
GCAACTGCAACCGGTGTACATTCTCAGATGCAGTTACAGGAGTCGGGCCCCGGACTGGTG
AAGCCTTCGGAAACCCTGTCCCTCACGTGCAGTGTGTCTGGTGCCTCCATAAGTGACAGTT
ACTGGAGCTGGATCCGGCGGTCCCCAGGGAAGGGACTTGAGTGGATTGGGTATGTCCACA
AAAGCGGCGACACAAATTACAGCCCCTCCCTCAAGAGTCGAGTCAACTTGTCGTTAGACA
CGTCCAAAAATCAGGTGTCCCTGAGCCTTGTGGCCGCGACCGCTGCGGACTCGGGCAAAT
ATTATTGCGCGAGAACACTGCACGGGAGGAGAATTTATGGAATCGTTGCCTTCAATGAGT
GGTTCACCTACTTCTACATGGACGTCTGGGGCAATGGGACTCAGGTCACCGTCTCCTCAGG
TAAGCTGGCTTTTTTCTTTCTGCACATTCCATTCTGAAACGGGATCGATTGGGAAATAAAC
TGTCTAGGGATCTCAGAGCCTTTAGGACAGATTATCTCCACATCTTTGAAAAACTAAGAAT
CTGTGTGATGGTGTTGGTGGAGTCCCTGGATGATGGGATAGGGACTTTGGAGGCTCATTTG
AAGAAGATGCTAAAACAATCCTATGGCTGGAGGGATAGTTGGGGCTGTAGTTGGAGATTT
TCAGTTTTTAGAATAAAAGTATTAGTTGTGGAATATACTTCAGGACCACCTCTGTGACAGC
ATTTATACAGTATCCGATGCATAGGGACAAAGAGTGGAGTGGGGCACTTTCTTTAGATTTG
TGAGGAATGTTCCGCACTAGATTGTTTAAAACTTCATTTGTTGGAAGGAGAGCTGTCTTAG
TGATTGAGTCAAGGGAGAAAGGCATCTAGCCTCGGTCTCAAAAGGGTAGTTGCTGTCTAG
AGAGGTCTGGTGGAGCCTGCAAAAGTCCAGCTTTCAAAGGAACACAGAAGTATGTGTATG
GAATATTAGAAGATGTTGCTTTTACTCTTAAGTTGGTTCCTAGGAAAAATAGTTAAATACT
GTGACTTTAAAATGTGAGAGGGTTTTCAAGTACTCATTTTTTTAAATGTCCAAAATTCTTGT
CAATCAGTTTGAGGTCTTGTTTGTGTAGAACTGATATTACTTAAAGTTTAACCGAGGAATG
GGAGTGAGGCTCTCTCATAACCTATTCAGAACTGACTTTTAACAATAATAAATTAAGTTTC
AAATATTTTTAAATGAATTGAGCAATGTTGAGTTGGAGTCAAGATGGCCTCGAGGAAT
3BNC60^{SI}, human (SEQ ID NO: 35)
GCATAGCTAGCGCTCTTCAACCACGGTCACCGTCTCCTCAGGTAAGAATGGCCACTCTAG
GGCCTTTGTTTTCTGCTACTGCCTGTGGGGTTTCCTGAGCATTGCAGGTTGGTCCTCGGGGC
ATGTTCCGAGGGGACCTGGGCGGACTGGCCAGGAGGGGATGGGCACTGGGGTGCCTTGAG
GATCTGGGAGCCTCTGACAGCGGGACGCAAGTAGTGAGGGCACTCAGAACGCCACTCAGC
CCCGACAGGCAGGGCACGAGGAGGCAGCTCCTCACCCTCCCTTTCTCTTTTGTCCTGCGGG
TCCTCAGGGAGTTAGAACTTGTTTATTGCAGCTTATAATGGTTACAAATAAAGCAATAGCA
TCACAAATTTCACAAATAAAGCATTTTTTTCACTGCATTCTAGTTGTGGTTTGTCCAAACTC
ATCAATGTATCTTATCATGTCTGGTAACGAGTGGCCACCTTTTCAGTGTTACCAGTGAGCT
CTGAGTGTTCCTAATGGGACCAGGATGGGTCTAGGTGCCTGCTCAATGTCAGAGACAGCA
ATGGTCCCACAAAAAACCCAGGTAATCTTTAGGCCAATAAAATGTGGGTTCACAGTGAGG
AGTGCATCCTGGGGTTGGGGTTTGTTCTGCAGCGGGAAGAGTGCTGTGCACAGAAAGCTT
AGAAATGGGGCAAGAGATGCTTTTCCTCAGGCAGGATTTAGGGCTTGGTCTCTCAGCATCC
CACACTTGTACAGCTGATGTGGCATCTGTGTTTTCTTTCTCATCCTAGATCAGGCTTTGAGC
TGTGAAATACCCTGCCTCATGCATATGCAAATAACCTGAGGTCTTCTGAGATAAATATAGA
TATATTGGTGCCCTGAGAGCATCACATAACAACCACATTCCTCCTCTGAAGAAGCCCCTGG
GAGCACAGCTCATCACCATGGGATGGTCATGTATCATCCTTTTTCTAGTAGCAACTGCAAC
CGGTGTACATTCTGACATCCAGATGACCCAGTCTCCATCCTCCCTGTCTGCATCTGTAGGA
GACAGAGTCACCATCACTTGCCAGGCGAGTCAGGACATTAGCAACTATTTAAATTGGTAT
CAGCAGAAACCAGGGAAAGCCCCTAAGCTCCTGATCTACGATGCATCCAATTTGGAAACA
GGGGTCCCATCAAGGTTCAGTGGAAGTGGATCTGGGACAGATTTTACTTTCACCATCAGCA
GCCTGCAGCCTGAAGATATTGCAACATATTACTGTCAACAGTATGAGTTTATCGGCCCTGG
GACCAAAGTGGATATCAAACGAACTGTGGCTGCACCATCTGTCTTCATCTTCCCGCCATCT
GATGAGCAGTTGAAATCTGGAACTGCCTCTGTTGTGTGCCTGCTGAATAACTTCTATCCCA
GAGAGGCCAAAGTACAGTGGAAGGTGGATAACGCTCTCCAAAGCGGTAACTCCCAGGAG
AGTGTCACAGAGCAGGACAGCAAGGACAGCACCTACAGCCTCAGCAGCACCCTGACGCTG
AGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTAGGCGGAAGCGGGGGTCAGG
AGCAACCAACTTTTCTCTGCTGAAGCAAGCCGGGGACGTAGAGGAAAACCCCGGACCCAT
GGGATGGTCATGTATCATCCTTTTTCTAGTAGCAACTGCAACCGGTGTACATTCTCAGGTC
CATTTGTCACAGTCTGGGGCAGCGGTGACGAAGCCCGGGGCCTCAGTGAGAGTCTCCTGC
GAGGCTTCCGGATACAAGATTAGTGACCACTTTATTCATTGGTGGCGACAGGCCCCAGGA
CAGGGCCTTCAGTGGGTGGGGTGGATCAATCCTAAGACTGGTCAGCCAAACAATCCTCGT
CAATTTCAGGGTAGAGTCAGTCTGACTCGACAGGCGTCGTGGGACTTTGACACATATTCCT
TTTACATGGACCTCAAGGCAGTAAGATCGGACGACACGGCCATTTATTTCTGTGCGCGACA
ACGCAGCGACTTTTGGGATTTCGACGTCTGGGGCAGCGGCACGCAGGTCACTGTCTCGTCA
GGTGAGTCCTCACAACCTCTCTCCTGCTTTAACTCTGAAGGGTTTTGCTGCTGGATTTTCCG
ATGCCTTTGGAAAATGGGACTCAGGTTGGGTGCGTCTGATGGAGTAACTGAGCCTGGGGG
CTTGGGGAGCCACATTTGGACGAGATGCCTGAACAAACCAGGGGTCTTAGTGATGGCTGA
GGAATGTGTCTCAGGAGCGGTGTCTGTAGGACTGCAAGATCGCTGCACAGCAGCGAATCG
TGAAATATTTTCTTTAGAATTATGAGGTGCGCTGTGTGTCAACCTGCATCTTAAATTCTTTA
TTGGCTGGAAAGAGAACTGTCGGAGTGGGTGAATCCAGCCAGGAGGGACGCGTAGCCCC
GGTCTTGATGAGAGCAGGGTTGGGGGCAGGGGTAGCCCAGAAACGGTGGCTGCCGTCCTG
ACAGGGGCTTAGGGAGGCTCCAGGACCTCAGTGCCTTGAAGCTGGTTTCCATGAGAAAAG
GATTGTTTATCTTAGGAGGCATGCTTACTGTTAAAAGACAGGATATGTTTGAAGTGGCTTC
TGAGAAAAATGGTTAAGAAAATTATGACTCGAGGAATT
10-1074, human (SEQ ID NO: 36)
GCATAGCTAGCGCTCTTCAACCACGGTCACCGTCTCCTCAGGTAAGAATGGCCACTCTAG
GGCCTTTGTTTTCTGCTACTGCCTGTGGGGTTTCCTGAGCATTGCAGGTTGGTCCTCGGGGC
ATGTTCCGAGGGGACCTGGGCGGACTGGCCAGGAGGGGATGGGCACTGGGGTGCCTTGAG
GATCTGGGAGCCTCTGACAGCGGGACGCAAGTAGTGAGGGCACTCAGAACGCCACTCAGC
CCCGACAGGCAGGGCACGAGGAGGCAGCTCCTCACCCTCCCTTTCTCTTTTGTCCTGCGGG
TCCTCAGGGAGTTAGAACTTGTTTATTGCAGCTTATAATGGTTACAAATAAAGCAATAGCA
TCACAAATTTCACAAATAAAGCATTTTTTTCACTGCATTCTAGTTGTGGTTTGTCCAAACTC
ATCAATGTATCTTATCATGTCTGGTAACGAGTGGCCACCTTTTCAGTGTTACCAGTGAGCT
CTGAGTGTTCCTAATGGGACCAGGATGGGTCTAGGTGCCTGCTCAATGTCAGAGACAGCA
ATGGTCCCACAAAAAACCCAGGTAATCTTTAGGCCAATAAAATGTGGGTTCACAGTGAGG
AGTGCATCCTGGGGTTGGGGTTTGTTCTGCAGCGGGAAGAGTGCTGTGCACAGAAAGCTT
AGAAATGGGGCAAGAGATGCTTTTCCTCAGGCAGGATTTAGGGCTTGGTCTCTCAGCATCC
CACACTTGTACAGCTGATGTGGCATCTGTGTTTTCTTTCTCATCCTAGATCAGGCTTTGAGC
TGTGAAATACCCTGCCTCATGCATATGCAAATAACCTGAGGTCTTCTGAGATAAATATAGA
TATATTGGTGCCCTGAGAGCATCACATAACAACCACATTCCTCCTCTGAAGAAGCCCCTGG
GAGCACAGCTCATCACCATGGGATGGTCATGTATCATCCTTTTTCTAGTAGCAACTGCAAC
CGGTGTACATTCTTCCTATGTGCGCCCGCTGTCAGTGGCCCTGGGGGAGACGGCCAGGATT
TCCTGTGGACGACAGGCCCTTGGAAGTAGAGCTGTTCAGTGGTATCAACATAGGCCAGGC
CAGGCCCCTATATTGCTCATTTATAATAATCAAGACCGGCCCTCAGGGATCCCTGAGCGAT
TCTCTGGCACCCCTGATATTAATTTTGGGACCAGGGCCACCCTGACCATCAGCGGGGTCGA
AGCCGGGGATGAAGCCGACTATTACTGTCACATGTGGGATAGTAGAAGTGGCTTCAGTTG
GTCTTTCGGCGGGGCGACCAGGCTGACCGTCCTACGAACTGTGGCTGCACCATCTGTCTTC
ATCTTCCCGCCATCTGATGAGCAGTTGAAATCTGGAACTGCCTCTGTTGTGTGCCTGCTGA
ATAACTTCTATCCCAGAGAGGCCAAAGTACAGTGGAAGGTGGATAACGCTCTCCAAAGCG
GTAACTCCCAGGAGAGTGTCACAGAGCAGGACAGCAAGGACAGCACCTACAGCCTCAGC
AGCACCCTGACGCTGAGCAAAGCAGACTACGAGAAACACAAAGTCTACGCCTGCGAAGTC
ACCCATCAGGGCCTGAGCTCGCCCGTCACAAAGAGCTTCAACAGGGGAGAGTGTAGGCGG
AAGCGGGGGTCAGGAGCAACCAACTTTTCTCTGCTGAAGCAAGCCGGGGACGTAGAGGA
AAACCCCGGACCCATGGGATGGTCATGTATCATCCTTTTTCTAGTAGCAACTGCAACCGGT
GTACATTCTCAGGTGCAGCTGCAGGAGTCGGGCCCAGGACTGGTGAAACCTTCGGAGACC
CTGTCCGTCACCTGCAGTGTCTCTGGAGATTCCATGAATAATTACTACTGGACTTGGATCC
GGCAGTCCCCCGGAAAGGGACTGGAGTGGATAGGCTATATCTCTGACAGAGAATCAGCGA
CTTACAACCCCTCCCTCAATAGTCGAGTCGTCATATCACGAGACACGTCGAAAAACCAATT
GTCCCTAAAATTAAACTCCGTCACCCCTGCGGACACGGCCGTCTATTACTGTGCGACAGCG
CGCCGAGGACAGAGGATTTATGGAGTGGTTTCCTTTGGAGAGTTCTTCTACTACTACTCCA
TGGACGTCTGGGGCAAGGGGACCACGGTCACCGTCTCCTCAGGTGAGTCCTCACAACCTC
TCTCCTGCTTTAACTCTGAAGGGTTTTGCTGCTGGATTTTCCGATGCCTTTGGAAAATGGGA
CTCAGGTTGGGTGCGTCTGATGGAGTAACTGAGCCTGGGGGCTTGGGGAGCCACATTTGG
ACGAGATGCCTGAACAAACCAGGGGTCTTAGTGATGGCTGAGGAATGTGTCTCAGGAGCG
GTGTCTGTAGGACTGCAAGATCGCTGCACAGCAGCGAATCGTGAAATATTTTCTTTAGAAT
TATGAGGTGCGCTGTGTGTCAACCTGCATCTTAAATTCTTTATTGGCTGGAAAGAGAACTG
TCGGAGTGGGTGAATCCAGCCAGGAGGGACGCGTAGCCCCGGTCTTGATGAGAGCAGGGT
TGGGGGCAGGGGTAGCCCAGAAACGGTGGCTGCCGTCCTGACAGGGGCTTAGGGAGGCTC
CAGGACCTCAGTGCCTTGAAGCTGGTTTCCATGAGAAAAGGATTGTTTATCTTAGGAGGCA
TGCTTACTGTTAAAAGACAGGATATGTTTGAAGTGGCTTCTGAGAAAAATGGTTAAGAAA
ATTATGACTCGAGGAATT

Murine Cell Culture

Mature, resting B cells were obtained from mouse spleens by forcing tissue through a 70 μm mesh into PBS containing 2% heat-inactivated fetal bovine serum (FBS). After ACK lysis for 3 min, untouched B cells were enriched using anti-CD43 magnetic beads (MACS) according to manufacturer's protocol (Miltenyi Biotec) obtaining >95% purity. 3.2×10⁷ cells/10 cm dish (Gibco) were cultured at 37° C. 5% CO2 in 10 mL mouse B cell medium consisting of RPMI-1640, supplemented with 10% heat-inactivated FBS, 10 mM HEPES, antibiotic-antimycotic (1×), 1 mM sodium pyruvate, 2 mM L-glutamine and 53 μM 2-mercaptoethanol (all from Gibco) and activated with 2 μg/mL anti-mouse RP105 clone RP/14 (produced in house or BD Pharmingen Cat. #562191).

NB-21 feeder cells (Kuraoka et al., 2016) were maintained in DMEM supplemented with 10% heat-inactivated FBS and antibiotic-antimycotic (1×). For co-culture, feeder cells were irradiated with 80 Gy and seeded simultaneously with B cells, 24 h after transfection, into B cell culture medium supplemented with 1 ng/mL recombinant mouse IL-4 (PeproTech Ca. #214-14) and 2 μg/mL anti-mouse RP105 clone RP/14.

Human Cell Culture

Leukapheresis samples of healthy human individuals were collected after signed informed consent in accordance with protocol TSC-0910 approved by the Rockefeller University Institutional Review Board (IRB). PBMCs were prepared, stored in liquid nitrogen, then thawed in a 37° C. water bath and resuspended in human B cell medium composed of RPMI-1640, supplemented with 10% heat-inactivated FBS or human serum, 10 mM HEPES, antibiotic-antimycotic (1×), 1 mM sodium pyruvate, 2 mM L-glutamine and 53 μM 2-mercaptoethanol (all from Gibco). B cells were isolated using EasySep human naïve B cell Enrichment Kit (Stemcell Cat. #19254) according to the manufacturer's instructions and cultured in the above medium supplemented with 2 μg/mL anti-human RP105 antibody clone MHR73-11 (BioLegend Cat. #312907).

RNP Preparation and Transfection

Per 100 μL transfection, 1 μL of 200 μM crRNA and 1 μL 200 μM tracrRNA in duplex buffer (all IDT) were mixed, denatured at 95° C. for 5 min, re-natured for 5 min at room temperature. 5.6 μL PBS and 2.4 μL 61 μM Cas9 V3 (IDT, Cat. #1081059) were added and incubated for 15-30 min. If required RNPs were mixed at the following ratios: 50% crIgH, 25% crIgK₁ and 25% crIgK₂ (mouse) or 50% crhIgH₃ and 50% crhIgK₃ (human). 4 μI, 100 μM electroporation enhancer in duplex buffer or 4 μL HDRT at 2.5 μg/μL were added to 10 μL mixed RNP and incubated for a further 1-2 min.

24 h after stimulation, activated mouse or human B cells were harvested, washed once in PBS and resuspended in Mouse B cell Nucleofector Solution with Supplement (murine B cells) or Primary Cell Nucleofector Solution 3 with Supplement (human B cells) prepared to the manufacturer's instructions (Lonza) at a concentration of 4-5×10⁶ cells/86 μL. 86 μL cells were added to the RNP/HPRT mix, gently mixed by pipetting and transferred into nucleofection cuvettes and electroporated using an Amaxa IIb machine setting Z-001 (murine B cells) or Amaxa 4D machine setting EH-140 (human B cells). Cells were immediately transferred into 6-well dishes containing 5 mL prewarmed mouse or human B cell medium supplemented with the relevant anti-RP105 antibody at 2 μg/mL and incubated at 37° C. 5% CO2 for 24 h before further processing.

TIDE Assay

Genomic DNA was extracted from 0.5-5×10⁵ cells by standard phenol/chloroform extraction 24-42 h after transfection. PCRs to amplify human or mouse Ig loci targeted by CRISPR-Cas9 were performed using Phusion Green Hot Start II High-Fidelity polymerase (Thermo Fisher Cat. #F-537L) and primers listed in Table 3. Thermocycler was set to 40 cycles, annealing at 65° C. for 30 s and extending at 72° C. for 30 s. PCR product size was verified by gel electrophoresis, bands gel-extracted and sent for Sanger sequencing (Genewiz) using the relevant PCR primers. ab1 files were analyzed using the TIDE web tool (tide.nki.nl) using samples receiving scramble or irrelevant HPRT-targeting crRNA as reference (Brinkman et al., 2014).

TABLE 3
Primers for TIDE analysis

| | SEQ | | | SEQ |
|---|---|---|---|---|
| Forward primer | ID | Reverse primer | | ID |
| 5′ to 3′ sequence | NO | 5′ to 3′ sequence | Comments | NO |
| TIDE 1 | 37 | TIDE 2 | for TIDE analysis | 38 |
| CCTGGCCCCATTGTTCCTTA | | GCGTCTCAGGACCTTTGTCT | mouse Igkc, product | |
| | | | 483 bp | |
| TIDE 3 | 39 | TIDE 4 | for TIDE analysis | 40 |
| AATGTCTGAGTTGCCCAGGG | | TGTCACAGAGGTGGTCCTGA | mouse J_{H}4 intron, | |
| | | | product 495 bp | |
| TIDE 5 | 41 | TIDE 6 | for TIDE analysis | 42 |
| ATGGCTGCAAAGAGCTCCAA | | GGAAAAAGGGTCAGAGGCCA | human IGKC, | |
| | | | product 638 bp | |
| TIDE 7 | 43 | TIDE 8 | for TIDE analysis | 44 |
| TGCCCTGTGATTATCCGCAA | | GAGCTGGAGGACCGCAATAG | human IGKC, | |
| | | | product 515 bp | |
| TIDE 9 | 45 | TIDE 10 | for TIDE analysis | 46 |
| GCCACTCTAGGGCCTTTGTT | | AGCTTCAAGGCACTGAGGTC | human J_{H}6 intron, | |
| | | | product 563 bp | |
| TIDE 11 | 47 | TIDE 12 | for TIDE analysis | 48 |
| CTACATGGACGTCTGGGGC | | CTGCTCTCATCAAGACCGGG | human J_{H}6 intron, | |
| | | | product 533 bp | |

Flow Cytometry

Mouse spleens were forced through a 70 μm mesh into FACS buffer (PBS containing 2% heat-inactivated FBS and 2 mM EDTA) and red blood cells were lysed in ACK lysing buffer (Gibco) for 3 min. Cultured cells were harvested by centrifugation. Then cells were washed and Fc-receptors blocked for 15 min on ice. Cells were stained for 20 min on ice with antibodies or reagents listed in Table 4 and depending on the stain, washed again and secondary stained for another 20 min on ice before acquisition on a BD LSRFortessa. Anti-idiotype 3BNC60^{SI} (iv8) produced as human IgG1/κ was detected with anti-human Igκ-BV421 on edited mouse B cells. GC B cells were gated as single/live, B220⁺, CD38⁻FAS⁺, GL7⁺, IgD⁻. Allotypic markers CD45.1 and CD45.2 were used to track adoptively transferred B cells.

TABLE 4
Flow cytometric reagents

| | Target | Antibody | | |
|---|---|---|---|---|
| Reagent | species | clone | Company/Source | Cat. # |
| CD16/32 | mouse | 2.4G2 | BD Biosciences | 7248907 |
| CD4-eF780 | mouse | RM4-5 | Thermo Fisher | 47-0042-82 |
| CD8a-eF780 | mouse | 53-6.7 | Thermo Fisher | 47-0081-82 |
| NK1.1-eF780 | mouse | PK136 | Thermo Fisher | 47-5941-82 |
| F4/80-eF780 | mouse | BM8 | Thermo Fisher | 47-4801-82 |
| LY6G | mouse | RB6-8C5 | Thermo Fisher | 47-5931-82 |
| (Gr1)-eF780 | | | | |
| IgG1-APC | mouse | A85-1 | BD Pharmingen | 560089 |
| CD95 | mouse | Jo2 | BD Biosciences | 557653 |
| (FAS)-PE-Cy7 | | | | |
| CD45.2-PE | mouse | 104 | BioLegend | 109808 |
| CD45.1-BV421 | mouse | A20 | BioLegend | 110732 |
| GL7-FITC | mouse | GL7 | BD Pharmingen | 553666 |
| IgD-BV786 | mouse | 11-26c.2a | BD Horizon | 563618 |
| CD45R/B220- | mouse/ | RA3-6B2 | BioLegend | 103244 |
| BV605 | human | | | |
| CD19-PECy7 | mouse | 6D5 | BioLegend | 115520 |
| IgM^{a}-FITC | mouse | DS-1 | BD Pharmingen | 553516 |
| IgM^{b}-PE | mouse | AF6-78 | BioLegend | 406208 |
| Ig light chain | mouse | RML-42 | BioLegend | 407306 |
| λ-APC | | | | |
| Ig light chain | mouse | 187.1 | BD Horizon | 562888 |
| κ-BV421 | | | | |
| IgM Fab-FITC | mouse | polyclonal | Jackson | 115-097-020 |
| | | | Immunoresearch | |
| Zombie NIR | N/A* | N/A | BioLegend | 423105 |
| Streptavidin-PE | N/A | N/A | BD Pharmingen | 554061 |
| Streptavidin- | N/A | N/A | BD Horizon | 563259 |
| BV421 | | | | |
| TM4 core-biotin | N/A | N/A | in house | N/A |
| | | | (McGuire 2014) | |
| 10mut-biotin | N/A | N/A | in house | N/A |
| | | | (Steichen 2016) | |
| anti-3BNC60^{SI} | N/A | Iv8 | in house, | N/A |
| idiotype | | | this publication | |
| Human Fc Block | human | N/A | BD Horizon | 564220 |
| Ig light chain | human | MHL38 | BioLegend | 316609 |
| λ-APC | | | | |
| CD19-PECy7 | human | SJ25C1 | BioLegend | 363011 |
| IgM-FITC | human | MHM88 | BioLegend | 314506 |
| IgD-BV785 | human | IA6-2 | BioLegend | 348241 |
| Ig light chain | human | MHK-49 | BioLegend | 316518 |
| κ-BV421 | | | | |

*N/A not available

Mice

C57BL/6J and B6.Igha (B6.Cg-Gpi1a Thy1a Igha/J) and B6.SJL were obtained from the Jackson Laboratory. Igha/b mice were obtained by intercrossing B6.Igha and B6.SJL mice. B1-8hi (Shih et al., 2002), 3BNC60SI (Dosenovic et al., 2018) and PGT121 (Escolano et al., 2016; Steichen et al., 2016) strains were generated and maintained in our laboratory on a C57BL/6J background. All experiments used age and sex-matched animals, littermates when possible. All experiments were performed with authorization from the Institutional Review Board and the Rockefeller University IACUC.

Cell Transfers and Immunizations

After culture, mouse B cells were harvested at the indicated time points and resuspended in mouse B cell medium without anti-RP105 antibody and rested for 2-3 h at 37° C., 5% CO2. Then cells were washed once in PBS and resuspended in 200 μL PBS/mouse containing the indicated number of initially transfected cells. 200 μL cell suspension/mouse were injected intravenously via the retroorbital sinus. Number of transferred, edited B cells was estimated as follows: Number of cells transfected×20% survival×0.15-0.4% transfection efficiency×50% handling/proliferation×5% transfer efficiency (Dosenovic et al., 2018). Mice were immunized intraperitoneally within 24 h after cell transfer with 200 μL containing 10 μg TM4 core (McGuire et al., 2014) or 10mut (Steichen et al., 2016) in PBS with 50% Ribi (Sigma Adjuvant system, Sigma Aldrich) prepared to the manufacturer's instructions. Mice were bled at the indicated time points from the submandibular vein. Blood was allowed to clot and then serum was separated by centrifugation for 10 min at 20817 g. Serum was stored at −20° C.

Anti-Idiotypic Antibody

IgG producing hybridomas were isolated from mice immunized with iGL-VRC01 at the Frederick Hutchinson Cancer Research Center Antibody Technology Resource. Hybridoma supernatants were screened against a matrix of inferred germline (iGL) VRCO1 class antibodies as well as irrelevant iGL-antibodies using a high throughput bead-based assay. One anti-idiotypic antibody, clone iv8, bound to additional VRC01 class antibodies, but it also bound to a chimeric antibody with an iGL-VRC01 class light chain paired with the 8ANC131 heavy chain (which is derived from VH1-46), and to 3BNC60^{SI}.

ELISAs

For determination of 3BNC60^{SI} levels, Corning 3690 half-well 96-well plates were coated overnight at 4° C. with 25 μL/well of 2 μg/mL human anti-3BNC60SI (clone iv8) IgG in PBS, then blocked with 150 μL/well PBS 5% skimmed milk for 2 h at room temperature (RT). Sera were diluted to 1:50 with PBS and 7 subsequent 3-fold dilutions. Recombinant 3BNC60SI (produced in house as mouse IgG1,κ) was diluted to 10 μg/mL in PBS followed by six 5-fold dilutions. Blocked plates were washed 4-times with PBS 0.05% Tween 20 and incubated with 25 μL diluted sera or antibody for 2 h at RT. Binding was revealed by either anti-mouse IgG-horseradish peroxidase (HRP) (Jackson ImmunoResearch, Cat. #115-035-071) or anti-mouse IgG1a-biotin (BD Pharmingen Cat. #553500) or anti-mouse IgG1b-biotin (BD Pharmingen Cat. #553533), all diluted 1:5000 in PBS, 25 μL/well and incubation for 1 h at RT. Biotinylated antibodies were subsequently incubated with Streptavidin-HRP (BD Pharmingen Cat. #554066), diluted 1:1000 in PBS, 25 μL/well for 30 min at RT. Plates were washed 4-times with PBS 0.05% Tween 20 in between steps and 6 times before addition of substrate using a Tecan Hydrospeed microplate washer. HRP activity was determined using TMB as substrate (Thermo Scientific Cat. #34021), adding 50 μL/well. Reactions were stopped with 50 μL/well 2 M H2SO4 and read at 450 and 570 nm on a FLUOstar Omega microplate reader (BMG Labtech). Data were analyzed with Microsoft Excel and GraphPad Prism 6.0. Absolute 3BNC60SI titers were interpolated from sigmoidal fits of recombinant 3BNC60^{SI} standard curves.

For determination of NP-binding antibodies the following modifications applied. Plates were coated with 10 μg/mL NP31-bovine serum albumin (BSA, Biosearch Technologies) and blocked with PBS 3% BSA. Sera, antibodies and secondary reagents were diluted in PBS 1% BSA 0.05% Tween20.

Neutralization Assays

Collected mouse serum was pooled and IgG purified using protein G Ab SpinTraps (GE Healthcare Cat. #28-4083-47) then concentrated and buffer-exchanged into PBS using Amicon Ultra 30K centrifugal filter units (Merck Millipore Cat. #UFC503024) according to the manufacturers' instructions.

TZM-b1 assays were performed as previously described (Montefiori, 2005). Neutralizing activity was calculated as a function of the reduction in Tat-inducible luciferase expression in the TZM-b1 reporter cell line in a single round of virus infection.

Additional Information:

FIG. 5 shows that B cells cultured and stimulated as for RNP transfection are able to participate in GCs and produce antibodies. FIG. 6 relates to the choice of murine IgH crRNAs and production of HDRTs. FIG. 7 provides data on murine B cell viability after transfection, Igh allelic exclusion and a promoterless HDRT to improve allelic exclusion. FIG. 8 relates to the choice of human crRNAs and viability of human B cells after transfection. FIG. 9 provides details and additional data of neutralization assays. Table 1 lists crRNA sequences. Table 2 contains annotated HDRT sequences. Table 3 contains primer sequences for TIDE assay and Table 4 details flow cytometric reagents.

References—This reference listing is not an indication that any particular reference is material to patentability:

Bar, K. J., M. C. Sneller, L. J. Harrison, J. S. Justement, E. T. Overton, M. E. Petrone, D. B. Salantes, C. A. Seamon, B. Scheinfeld, R. W. Kwan, G. H. Learn, M. A. Proschan, E. F. Kreider, J. Blazkova, M. Bardsley, E. W. Refsland, M. Messer, K. E. Clarridge, N. B. Tustin, P. J. Madden, K. Oden, S. J. O'Dell, B. Jarocki, A. R. Shiakolas, R. L. Tressler, N. A. Doria-Rose, R. T. Bailer, J. E. Ledgerwood, E. V. Capparelli, R. M. Lynch, B. S. Graham, S. Moir, R. A. Koup, J. R. Mascola, J. A. Hoxie, A. S. Fauci, P. Tebas, and T. W. Chun. 2016. Effect of HIV Antibody VRC01 on Viral Rebound after Treatment Interruption. N Engl J Med 375:2037-2050.

Briney, B., D. Sok, J. G. Jardine, D. W. Kulp, P. Skog, S. Menis, R. Jacak, O. Kalyuzhniy, N. de Val, F. Sesterhenn, K. M. Le, A. Ramos, M. Jones, K. L. Saye-Francisco, T. R. Blane, S. Spencer, E. Georgeson, X. Hu, G. Ozorowski, Y. Adachi, M. Kubitz, A. Sarkar, I. A. Wilson, A. B. Ward, D. Nemazee, D. R. Burton, and W. R. Schief. 2016. Tailored Immunogens Direct Affinity Maturation toward HIV Neutralizing Antibodies. Cell 166:1459-1470.e1411.

Brinkman, E. K., T. Chen, M. Amendola, and B. van Steensel. 2014. Easy quantitative assessment of genome editing by sequence trace decomposition. Nucleic Acids Res 42:e168. Caskey, M., F. Klein, J. C. Lorenzi, M. S. Seaman, A. P. West, N. Buckley, G. Kremer, L. Nogueira, M. Braunschweig, J. F. Scheid, J. A. Horwitz, I. Shimeliovich, S. Ben-Avraham, M. Witmer-Pack, M. Platten, C. Lehmann, L. A. Burke, T. Hawthorne, R. J. Gorelick, B. D. Walker, T. Keler, R. M. Gulick, G. Fakenheuer, S. J. Schlesinger, and M. C. Nussenzweig. 2015. Viraemia suppressed in HIV-1-infected humans by broadly neutralizing antibody 3BNC117. Nature 522:487-491.

Caskey, M., T. Schoofs, H. Gruell, A. Settler, T. Karagounis, E. F. Kreider, B. Murrell, N. Pfeifer, L. Nogueira, T. Y. Oliveira, G. H. Learn, Y. Z. Cohen, C. Lehmann, D. Gillor, I. Shimeliovich, C. Unson-O'Brien, D. Weiland, A. Robles, T. Kummerle, C. Wyen, R. Levin, M. Witmer-Pack, K. Eren, C. Ignacio, S. Kiss, A. P. West, H. Mouquet, B. S. Zingman, R. M. Gulick, T. Keler, P. J. Bjorkman, M. S. Seaman, B. H. Hahn, G. Fakenheuer, S. J. Schlesinger, M. C. Nussenzweig, and F. Klein. 2017. Antibody 10-1074 suppresses viremia in HIV-1-infected individuals. Nat Med 23:185-191.

Cebra, J. J., J. E. Colberg, and S. Dray. 1966. Rabbit lymphoid cells differentiated with respect to alpha-, gamma-, and mu-heavy polypeptide chains and to allotypic markers Aa1 and Aa2. J Exp Med 123:547-558.

Dosenovic, P., E. E. Kara, A. K. Pettersson, A. T. McGuire, M. Gray, H. Hartweger, E. S. Thientosapol, L. Stamatatos, and M. C. Nussenzweig. 2018. Anti-HIV-1 B cell responses are dependent on B cell precursor frequency and antigen-binding affinity. Proc Natl Acad Sci USA 115:4743-4748.

Dosenovic, P., L. von Boehmer, A. Escolano, J. Jardine, N. T. Freund, A. D. Gitlin, A. T. McGuire, D. W. Kulp, T. Oliveira, L. Scharf, J. Pietzsch, M. D. Gray, A. Cupo, M. J. van Gils, K. H. Yao, C. Liu, A. Gazumyan, M. S. Seaman, P. J. Bjorkman, R. W. Sanders, J. P. Moore, L. Stamatatos, W. R. Schief, and M. C. Nussenzweig. 2015. Immunization for HIV-1 Broadly Neutralizing Antibodies in Human Ig Knockin Mice. Cell 161:1505-1515.

Escolano, A., P. Dosenovic, and M. C. Nussenzweig. 2017. Progress toward active or passive HIV-1 vaccination. J Exp Med 214:3-16.

Escolano, A., J. M. Steichen, P. Dosenovic, D. W. Kulp, J. Golijanin, D. Sok, N. T. Freund, A. D. Gitlin, T. Oliveira, T. Araki, S. Lowe, S. T. Chen, J. Heinemann, K. H. Yao, E. Georgeson, K. L. Saye-Francisco, A. Gazumyan, Y. Adachi, M. Kubitz, D. R. Burton, W. R. Schief, and M. C. Nussenzweig. 2016. Sequential Immunization Elicits Broadly Neutralizing Anti-HIV-1 Antibodies in Ig Knockin Mice. Cell 166:1445-1458.e1412.

Eyquem, J., J. Mansilla-Soto, T. Giavridis, S. J. van der Stegen, M. Hamieh, K. M. Cunanan, A. Odak, M. Gillen, and M. Sadelain. 2017. Targeting a CAR to the TRAC locus with CRISPR/Cas9 enhances tumour rejection. Nature 543:113-117.

Freitag, J., S. Heink, E. Roth, J. Wittmann, H. M. Jäck, and T. Kamradt. 2014. Towards the generation of B-cell receptor retrogenic mice. PLoS One 9:e109199.

Jacobsen, J. T., L. Mesin, S. Markoulaki, A. Schiepers, C. B. Cavazzoni, D. Bousbaine, R. Jaenisch, and G. D. Victora. 2018. One-step generation of monoclonal B cell receptor mice capable of isotype switching and somatic hypermutation. J Exp Med 215:2686-2695.

Kraus, M., M. B. Alimzhanov, N. Raj ewsky, and K. Raj ewsky. 2004. Survival of resting mature B lymphocytes depends on BCR signaling via the Igalpha/beta heterodimer. Cell 117:787-800.

Kuraoka, M., A. G. Schmidt, T. Nojima, F. Feng, A. Watanabe, D. Kitamura, S. C. Harrison, T. B. Kepler, and G. Kelsoe. 2016. Complex Antigens Drive Permissive Clonal Selection in Germinal Centers. Immunity 44:542-552.

Kwong, P. D., and J. R. Mascola. 2018. HIV-1 Vaccines Based on Antibody Identification, B Cell Ontogeny, and Epitope Structure. Immunity 48:855-871.

Lam, K. P., R. Kuhn, and K. Rajewsky. 1997. In vivo ablation of surface immunoglobulin on mature B cells by inducible gene targeting results in rapid cell death. Cell 90:1073-1083.

Ledgerwood, J. E., E. E. Coates, G. Yamshchikov, J. G. Saunders, L. Holman, M. E. Enama, A. DeZure, R. M. Lynch, I. Gordon, S. Plummer, C. S. Hendel, A. Pegu, M. Conan-Cibotti, S. Sitar, R. T. Bailer, S. Narpala, A. McDermott, M. Louder, S. O'Dell, S. Mohan, J. P. Pandey, R. M. Schwartz, Z. Hu, R. A. Koup, E. Capparelli, J. R. Mascola, B. S. Graham, and V. S. Team. 2015. Safety, pharmacokinetics and neutralization of the broadly neutralizing HIV-1 human monoclonal antibody VRC01 in healthy adults. Clin Exp Immunol 182:289-301. Lim, W. A., and C. H. June. 2017. The Principles of Engineering Immune Cells to Treat Cancer. Cell 168:724-740.

Lynch, R. M., E. Boritz, E. E. Coates, A. DeZure, P. Madden, P. Costner, M. E. Enama, S. Plummer, L. Holman, C. S. Hendel, I. Gordon, J. Casazza, M. Conan-Cibotti, S. A. Migueles, R. Tressler, R. T. Bailer, A. McDermott, S. Narpala, S. O'Dell, G. Wolf, J. D. Lifson, B. A. Freemire, R. J. Gorelick, J. P. Pandey, S. Mohan, N. Chomont, R. Fromentin, T. W. Chun, A. S. Fauci, R. M. Schwartz, R. A. Koup, D. C. Douek, Z. Hu, E. Capparelli, B. S. Graham, J. R. Mascola, J. E. Ledgerwood, and V. S. Team. 2015. Virologic effects of broadly neutralizing antibody VRC01 administration during chronic HIV-1 infection. Sci Transl Med 7:319ra206.

Mascola, J. R., M. G. Lewis, G. Stiegler, D. Harris, T. C. VanCott, D. Hayes, M. K. Louder, C. R. Brown, C. V. Sapan, S. S. Frankel, Y. Lu, M. L. Robb, H. Katinger, and D. L. Birx. 1999. Protection of Macaques against pathogenic simian/human immunodeficiency virus 89.6PD by passive transfer of neutralizing antibodies. J Virol 73:4009-4018.

McCoy, L. E., A. F. Quigley, N. M. Strokappe, B. Bulmer-Thomas, M. S. Seaman, D. Mortier, L. Rutten, N. Chander, C. J. Edwards, R. Ketteler, D. Davis, T. Verrips, and R. A. Weiss. 2012. Potent and broad neutralization of HIV-1 by a llama antibody elicited by immunization. J Exp Med 209:1091-1103.

McGuire, A. T., A. M. Dreyer, S. Carbonetti, A. Lippy, J. Glenn, J. F. Scheid, H. Mouquet, and L. Stamatatos. 2014. HIV antibodies. Antigen modification regulates competition of broad and narrow neutralizing HIV antibodies. Science 346:1380-1383.

McGuire, A. T., M. D. Gray, P. Dosenovic, A. D. Gitlin, N. T. Freund, J. Petersen, C. Correnti, W. Johnsen, R. Kegel, A. B. Stuart, J. Glenn, M. S. Seaman, W. R. Schief, R. K. Strong, M. C. Nussenzweig, and L. Stamatatos. 2016. Specifically modified Env immunogens activate B-cell precursors of broadly neutralizing HIV-1 antibodies in transgenic mice. Nat Commun 7:10618.

Mendoza, P., H. Gruell, L. Nogueira, J. A. Pai, A. L. Butler, K. Millard, C. Lehmann, I. Suárez, T. Y. Oliveira, J. C. C. Lorenzi, Y. Z. Cohen, C. Wyen, T. Kümmerle, T. Karagounis, C. L. Lu, L. Handl, C. Unson-O'Brien, R. Patel, C. Ruping, M. Schlotz, M. Witmer-Pack, I. Shimeliovich, G. Kremer, E. Thomas, K. E. Seaton, J. Horowitz, A. P. West, P. J. Bjorkman, G. D. Tomaras, R. M. Gulick, N. Pfeifer, G. Fätkenheuer, M. S. Seaman, F. Klein, M. Caskey, and M. C. Nussenzweig. 2018. Combination therapy with anti-HIV-1 antibodies maintains viral suppression. Nature 561:479-484.

Miura, Y., R. Shimazu, K. Miyake, S. Akashi, H. Ogata, Y. Yamashita, Y. Narisawa, and M. Kimoto. 1998. RP105 is associated with MD-1 and transmits an activation signal in human B cells. Blood 92:2815-2822.

Montefiori, D. C. 2005. Evaluating neutralizing antibodies against HIV, SIV, and SHIV in luciferase reporter gene assays. Curr Protoc Immunol Chapter 12:Unit 12.11.

Mouquet, H., L. Scharf, Z. Euler, Y. Liu, C. Eden, J. F. Scheid, A. Halper-Stromberg, P. N. Gnanapragasam, D. I. Spencer, M. S. Seaman, H. Schuitemaker, T. Feizi, M. C. Nussenzweig, and P. J. Bjorkman. 2012. Complex-type N-glycan recognition by potent broadly neutralizing HIV antibodies. Proc Natl Acad Sci USA 109:E3268-3277.

Nishimura, Y., and M. A. Martin. 2017. Of Mice, Macaques, and Men: Broadly Neutralizing Antibody Immunotherapy for HIV-1. Cell Host Microbe 22:207-216. Nussenzweig, M. C., A. C. Shaw, E. Sinn, D. B. Danner, K. L. Holmes, H. C. Morse, and P. Leder. 1987. Allelic exclusion in transgenic mice that express the membrane form of immunoglobulin mu. Science 236:816-819.

Parren, P. W., P. A. Marx, A. J. Hessell, A. Luckay, J. Harouse, C. Cheng-Mayer, J. P. Moore, and D. R. Burton. 2001. Antibody protects macaques against vaginal challenge with a pathogenic R5 simian/human immunodeficiency virus at serum levels giving complete neutralization in vitro. J Virol 75:8340-8347.

Pernis, B., G. Chiappino, A. S. Kelus, and P. G. Gell. 1965. Cellular localization of immunoglobulins with different allotypic specificities in rabbit lymphoid tissues. J Exp Med 122:853-876.

Roth, T. L., C. Puig-Saus, R. Yu, E. Shifrut, J. Carnevale, P. J. Li, J. Hiatt, J. Saco, P. Krystofinski, H. Li, V. Tobin, D. N. Nguyen, M. R. Lee, A. L. Putnam, A. L. Ferris, J. W. Chen, J. N. Schickel, L. Pellerin, D. Carmody, G. Alkorta-Aranburu, D. Del Gaudio, H. Matsumoto, M. Morell, Y. Mao, M. Cho, R. M. Quadros, C. B. Gurumurthy, B. Smith, M. Haugwitz, S. H. Hughes, J. S. Weissman, K. Schumann, J. H. Esensten, A. P. May, A. Ashworth, G. M. Kupfer, S. A. W. Greeley, R. Bacchetta, E. Meffre, M. G. Roncarolo, N. Romberg, K. C. Herold, A. Ribas, M. D. Leonetti, and A. Marson. 2018. Reprogramming human T cell function and specificity with non-viral genome targeting. Nature 559:405-409.

Sadelain, M., I. Rivière, and S. Riddell. 2017. Therapeutic T cell engineering. Nature 545:423-431.

Scheid, J. F., J. A. Horwitz, Y. Bar-On, E. F. Kreider, C. L. Lu, J. C. Lorenzi, A. Feldmann, M. Braunschweig, L. Nogueira, T. Oliveira, I. Shimeliovich, R. Patel, L. Burke, Y. Z. Cohen, S. Hadrigan, A. Settler, M. Witmer-Pack, A. P. West, B. Juelg, T. Keler, T. Hawthorne, B. Zingman, R. M. Gulick, N. Pfeifer, G. H. Learn, M. S. Seaman, P. J. Bjorkman, F. Klein, S. J. Schlesinger, B. D. Walker, B. H. Hahn, M. C. Nussenzweig, and M. Caskey. 2016. HIV-1 antibody 3BNC117 suppresses viral rebound in humans during treatment interruption. Nature 535:556-560.

Schoofs, T., F. Klein, M. Braunschweig, E. F. Kreider, A. Feldmann, L. Nogueira, T. Oliveira, J. C. Lorenzi, E. H. Parrish, G. H. Learn, A. P. West, P. J. Bjorkman, S. J. Schlesinger, M. S. Seaman, J. Czartoski, M. J. McElrath, N. Pfeifer, B. H. Hahn, M. Caskey, and M. C. Nussenzweig. 2016. HIV-1 therapy with monoclonal antibody 3BNC117 elicits host immune responses against HIV-1. Science 352:997-1001.

Shibata, R., T. Igarashi, N. Haigwood, A. Buckler-White, R. Ogert, W. Ross, R. Willey, M. W. Cho, and M. A. Martin. 1999. Neutralizing antibody directed against the HIV-1 envelope glycoprotein can completely block HIV-1/SIV chimeric virus infections of macaque monkeys. Nat Med 5:204-210.

Shih, T. A., M. Roederer, and M. C. Nussenzweig. 2002. Role of antigen receptor affinity in T cell-independent antibody responses in vivo. Nat Immunol 3:399-406.

Shingai, M., O. K. Donau, R. J. Plishka, A. Buckler-White, J. R. Mascola, G. J. Nabel, M. C. Nason, D. Montefiori, B. Moldt, P. Poignard, R. Diskin, P. J. Bjorkman, M. A. Eckhaus, F. Klein, H. Mouquet, J. C. Cetrulo Lorenzi, A. Gazumyan, D. R. Burton, M. C. Nussenzweig, M. A. Martin, and Y. Nishimura. 2014. Passive transfer of modest titers of potent and broadly neutralizing anti-HIV monoclonal antibodies block SHIV infection in macaques. J Exp Med 211:2061-2074. Sok, D., and D. R. Burton. 2018. Recent progress in broadly neutralizing antibodies to HIV. Nat Immunol 19:1179-1188.

Sok, D., K. M. Le, M. Vadnais, K. L. Saye-Francisco, J. G. Jardine, J. L. Torres, Z. T. Berndsen, L. Kong, R. Stanfield, J. Ruiz, A. Ramos, C. H. Liang, P. L. Chen, M. F. Criscitiello, W. Mwangi, I. A. Wilson, A. B. Ward, V. V. Smider, and D. R. Burton. 2017. Rapid elicitation of broadly neutralizing antibodies to HIV by immunization in cows. Nature 548:108-111. Steichen, J. M., D. W. Kulp, T. Tokatlian, A. Escolano, P. Dosenovic, R. L. Stanfield, L. E. McCoy, G. Ozorowski, X. Hu, O. Kalyuzhniy, B. Briney, T. Schiffner, F. Garces, N. T. Freund, A. D. Gitlin, S. Menis, E. Georgeson, M. Kubitz, Y. Adachi, M. Jones, A. A. Mutafyan, D. S. Yun, C. T. Mayer, A. B. Ward, D. R. Burton, I. A. Wilson, D. J. Irvine, M. C. Nussenzweig, and W. R. Schief. 2016. HIV Vaccine Design to Target Germline Precursors of Glycan-Dependent Broadly Neutralizing Antibodies. Immunity 45:483-496.

Tian, M., C. Cheng, X. Chen, H. Duan, H. L. Cheng, M. Dao, Z. Sheng, M. Kimble, L. Wang, S. Lin, S. D. Schmidt, Z. Du, M. G. Joyce, Y. Chen, B. J. DeKosky, E. Normandin, E. Cantor, R. E. Chen, N. A. Doria-Rose, Y. Zhang, W. Shi, W. P. Kong, M. Choe, A. R. Henry, F. Laboune, I. S. Georgiev, P. Y. Huang, S. Jain, A. T. McGuire, E. Georgeson, S. Menis, D. C. Douek, W. R. Schief, L. Stamatatos, P. D. Kwong, L. Shapiro, B. F. Haynes, J. R. Mascola, and F. W. Alt. 2016. Induction of HIV Neutralizing Antibody Lineages in Mice with Diverse Precursor Repertoires. Cell 166:1471-1484.e1418.

Voss, J. E., A. Gonzalez-Martin, R. Andrabi, R. P. Fuller, B. Murrell, L. E. McCoy, K. Porter, D. Huang, W. Li, D. Sok, K. Le, B. Briney, M. Chateau, G. Rogers, L. Hangartner, A. J. Feeney, D. Nemazee, P. Cannon, and D. Burton. 2019. Reprogramming the antigen specificity of B cells using genome-editing technologies. Elife 8:

Yoshimi, K., Y. Kunihiro, T. Kaneko, H. Nagahora, B. Voigt, and T. Mashimo. 2016. ssODN-mediated knock-in with CRISPR-Cas for large genomic regions in zygotes. Nat Commun 7:10431.

Although the subject matter of this disclosure has been described above in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

What is claimed is:

1. A method for modifying one or more primary B cells to provide one or more modified primary B cells, wherein the modified primary B cells maintain allelic exclusion and can participate in a humoral immune response when introduced into a mammal, and wherein the modified primary B cells produce heterologous antibodies that bind with specificity to a distinct epitope, the method comprising introducing into the one or more B cells:
1) a CAS enzyme or polynucleotide encoding the CAS enzyme;
2) a first and second guide RNA (gRNA), and optionally a third gRNA, wherein the first gRNA is targeted to an endogenous heavy chain locus, the second gRNA is targeted to a κ-light chain locus, and wherein if included, a third gRNA is targeted to a λ-light chain locus;
3) a ssDNA homology directed repair template (HDRT) comprising:
a) a first homology arm;
b) a splice acceptor site;
c) nucleotides from constant mu (Cμ) exon 1;
d) a sequence encoding a first amino acid linker sequence;
e) a sequence encoding a first self-cleaving amino acid sequence;
f) a sequence encoding leader, variable, and joining regions (VJ) of the heterologous antibody light chain;
g) a sequence encoding a kappa constant region (C_{κ});
h) a sequence encoding a protease-cleavage site;
i) a sequence encoding a second amino acid linker sequence;
j) a sequence encoding a second self-cleaving amino acid sequence;
k) a sequence encoding leader, variable, diversity, and joining regions (VDJ) of the heterologous heavy antibody chain;
l ) an intron splice donor site; and
m) a second homology arm;
and wherein the HDRT integrates into a suitable chromosomal locus targeted by the first and second homology arms in the one or more primary B cells to provide the one or more modified primary B cells, and wherein the one or more modified primary B cells produce the heterologous antibody that comprises at least the VJ and VDJ regions.

2. The method of claim 1, wherein at least one of the following is true:
i) no promoter is included in the HDRT;
ii) the primary B cells are human B cells;
iii) only two nucleotides from the Cμ exon 1 are included in the HDRT;
iv) the first or second self-cleaving amino acid sequences comprise a T2A sequence or a P2A sequence;
v) the first or second amino acid linker sequences, or both, are GSG-linker sequences;
vi) the protease cleavage site is a furin-cleavage site;
vii) the suitable chromosomal locus is a human IGKC exon and/or a human IGHJ6 intron and/or a human IgLC locus;
viii) the CAS enzyme and the guide RNAs are introduced into the primary B cell as a ribonucleotide protein complex;
ix) if a plurality of primary B cells are made according to claim 1, more of the primary B cells will be λ-B cell receptor positive primary B cells than κ-B cell receptor positive primary B cells; or the amount of λ-B cell receptor positive primary B cells are reduced;
x) steps 1)-3) are performed without using a viral delivery vector;
xi) the CAS enzyme is a Cas9 enzyme.

3. The method of claim 2, wherein all of i)-xi) are true.

4. The method of claim 1, wherein the sequence encoding the leader, variable, and joining regions (VJ) of the heterologous antibody light chain and the sequence encoding the variable, diversity, and joining regions (VDJ) of the heterologous heavy antibody chain are expressed by the one or more of modified primary B cells and form functional antibodies comprising said VJ and VDJ regions.

5. The method of claim 4, wherein the functional antibodies are anti-viral antibodies.

6. The method of claim 5, wherein the functional antibodies comprise broadly neutralizing antibodies.

7. The method of claim 6, wherein the broadly neutralizing antibodies recognize an epitope comprised by an antigen expressed by Human Immunodeficiency Virus.

8. The method of claim 2, wherein the sequence encoding the leader, variable, and joining regions (VJ) of the heterologous antibody light chain and the sequence encoding the variable, diversity, and joining regions (VDJ) of the heterologous heavy antibody chain are expressed by the one or more of modified primary B cells and form functional antibodies comprising said VJ and VDJ regions.

9. The method of claim 8, wherein the functional antibodies are anti-viral antibodies.

10. The method of claim 9, wherein the functional antibodies comprise broadly neutralizing antibodies.

11. The method of claim 10, wherein the broadly neutralizing antibodies recognize an epitope expressed by Human Immunodeficiency Virus.

12. A method comprising administering modified primary B cells made according to claim 1 to an individual in need thereof.

13. The method of claim 12, further comprising vaccinating the individual with an antigen comprising an epitope to which heterologous antibodies produced by the modified primary B cells bind with specificity to thereby stimulate production of the heterologous antibodies by the modified primary B cells.

14. The method of claim 13, wherein the individual is in need of treatment for a condition that is correlated with the presence of the antigen comprising the epitope to which the heterologous antibodies expressed by the modified primary B cells bind with specificity, wherein the modified primary B cells produce the heterologous antibodies that bind to said epitope.

15. The method of claim 14, wherein the heterologous antibodies bind with specificity to a single distinct epitope of an antigen expressed by a pathogen or a cancer cell.

16. The method of claim 15, wherein the pathogen is a virus, and wherein optionally the heterologous antibodies are neutralizing for the virus.

17. The method of claim 16, wherein the antibodies are the neutralizing antibodies and bind with specificity to an epitope on a Human Immunodeficiency Virus (HIV).

18. A composition comprising modified primary B cells made according to the method of claim 1.

19. Heterologous antibodies isolated from primary B cells made according to the method of claim 1.

20. A mixture of distinct modified primary B cells made according to claim 1, wherein the distinct modified primary B cells produce heterologous antibodies that bind to distinct epitopes.
